(19) United States
(12) Patent Application Publication
(10) Pub. No.: US 2023/0023609 A1
(43) Pub. Date: Jan. 26, 2023

(54) SYSTEMS AND METHODS FOR ANIMATING A SIMULATED FULL LIMB FOR AN AMPUTEE IN VIRTUAL REALITY

(71) Applicant: PENUMBRA, INC., Alameda, CA (US)

(72) Inventors: Hans Peter Winold, Berkeley, CA (US); Andrew Taylor Langley, Alameda, CA (US)

(21) Appl. No.: 17/382,788

(22) Filed: Jul. 22, 2021

(51) Int. Cl. G06T 13/40; G06T 15/00; A61M 21/00; A63B 71/06

(52) U.S. Cl. CPC G06T 13/40; G06T 15/00; A61M 21/00; A63B 71/0622; G06T 2213/12; A61M 2021/005; A63B 2071/0636; A63G 31/16

(57) ABSTRACT

A system and method for generating simulated full limb animations in real time based on sensor and tracking data. A computing environment for receiving and processing tracking data from one or more sensors, for mapping tracking data onto a 3D model having a skeletal hierarchy and a surface topology, and for rendering an avatar for display in virtual reality. A method for animating a full-bodied avatar from tracking data collected from an amputee. A means for determining, predicting, or modulating movements an amputee intends to make with his or her simulated full limb. A modified inverse kinematics method for arbitrarily and artificially overriding a position and orientation of a tracked end effector. Synchronous virtual reality therapeutic activities with predefined movement patterns that may modulate animations.

### BACKGROUND OF THE DISCLOSURE

The present disclosure relates generally to the field of animation methods. More particularly, the disclosure describes herein relates to methods for animating movements of a simulated full limb for display in a virtual environment to an amputee.

### SUMMARY OF THE DISCLOSURE

Virtual reality (VR) systems may be used in various applications, including therapeutic activities and games, to assist patients with their rehabilitation and recovery from illness or injury including patients with one or more amputated limbs. An amputee's participation in, e.g., physical and neurocognitive therapy activities may help to improve, e.g., pain management, sensory complications, coordination, range of motion, mobility, flexibility, endurance, strength, etc. Animating a patient as an avatar for therapeutic VR activities in a virtual world can improve engagement and immersion in therapy. Likewise, animating a virtual simulated full limb in place of an amputated limb can aid VR therapy for amputees. Animating a virtual full limb for a therapy patient, according to the embodiments discussed below, may help reduce issues known to affect amputees.

### Phantom Limb Pain

A person who has lost a limb may continue to feel some sensations in the limb even after it is gone. This often manifests as a feeling and/or illusion in the amputee's mind that a limb is still there, e.g., called a “phantom limb.” For example, an amputee may feel sensations of touch, pressure, pain, itchiness, tingling, and/or temperature in their missing “phantom” arm or leg that is missing in reality. These sensations may conflict with visual perception and may often lead to the perception of localized excruciating pain at the point of loss or the missing limb, e.g., commonly known as phantom limb pain. Amputees may also experience sensations that their phantom limb is functioning, despite not seeing or having anything at the site of the sensation. For instance, an amputee may feel sensations that their phantom limb is telescoping (e.g., a limb is gradually shortening), moving of its own accord, or paralyzed in an uncomfortable position, such as a tightly clenched fist. These sensations may also conflict with visual perception and may hinder control over a remaining portion of the limb. Providing a match between expected and actual sensory feedback may be a key to alleviating phantom limb pain and related sensations. For example, experiments have shown that providing a visual representation of a phantom limb, over which an amputee has volitional control, can alleviate phantom limb pain. These visual representations can show that a phantom limb is not in pain or paralyzed in an uncomfortable position, which has been shown to counteract the negative sensations associated with phantom limbs.

### Working to Alleviate Phantom Limb Pain

A visual representation of an amputee's missing limb may be provided in many ways. Visual representations may be generated with mirrors, robotics, virtual reality, or augmented reality to provide phantom limb pain therapy. These therapies typically attempt to normalize the cortical representation of the missing or phantom limb and improve the correspondence between actual and predicted sensory feedback.

One traditional treatment for phantom limb pain is mirror therapy. For a patient missing (part of) a leg, sitting down with the intact leg extended and placing a long mirror between the legs. Mirror therapy for upper body parts (e.g., arms) typical utilizes a box with a mirror in the middle into which an amputee places each of their intact limb and their remaining limb in respective portions. The mirror (e.g., in the box) enables an amputee to see a mirror image of their complete limb where a (simulated) phantom limb should be. In this way, the mirror can fool a patient's brain into thinking the mirror image is the missing limb. An amputee is then instructed to move their limbs in synchronicity to match the reflected motion to provide a match between expected and actual visual feedback during volitional movements. For instance, with a missing left hand or arm, the right arm movements are reflected on the left side. Seeing the missing limb move according to an amputee's volition establishes a sense of control over a mirror-created full limb and may reduce phantom limb pain.

Although mirror therapy is relatively inexpensive and provides the benefit of a perfect visual image, the illusion is often not compelling or engaging to users. With an amputee's remaining portion of a left arm behind the mirror, a reflected right arm must perform the actions intended for the left at the same time. For instance, an amputee cannot independently control the mirrored limb because the mirror can only provide visualizations of movements that are symmetric to the intact limb. This severely limits the variety of movements that can be performed and thereby limits amputee engagement. For instance, crossing arms or legs is not feasible.

Other approaches may involve robotics and virtual reality. Robotics and virtual reality may offer a more sophisticated approach than the mirror, which can expand on the concept of mirror image therapy in a more engaging manner. By tracking the intact limb, the robotics (or VR system) can replicate mirrored movement on the simulated limb. These approaches may allow a bit more movement than a mirror box. For instance, a patient missing a right hand may move his right arm freely and a simulated right hand may be controlled by a left hand that is stationary. The development of these techniques, however, requires greater investment and the tools themselves are often expensive. This is especially true in the case of robotic devices, which can cost upwards of $25,000. Robotic therapy for all amputees may not be feasible. While VR may be less expensive (e.g., $300-$1,000), the basis of current VR applications for amputees in mirror therapy still leaves many movements restricted to mirroring intact limbs, which may lead to mixed results regarding engagement and follow through.

Another approach may use myoelectric techniques with, e.g., robotic and/or virtual reality treatments of phantom limb pain. For instance, with myoelectric techniques, electrodes may be placed on an amputee's residual limb to collect muscle electromyography (EMG) signals. The residual limb is often, respectfully, referred to as a “stump.” EMG signals from the electrodes on the stump are collected while an amputee systematically attempts to instruct the missing limb to perform specific actions (e.g., making a fist, splaying the fingers, etc.), which establishes training data for use by a learning algorithm. Once sufficient training data is collected, the learned algorithm may be able to predict a user's body commands based on the EMG signals and then provide a representation of those controls in the form a robotic limb moving or a virtual limb moving.

In contrast to mirror therapy or virtual reality systems where representations of the missing limb are typically controlled by the intact limb alone, an EMG system uses signals from the damaged limb itself, which enables wider ranges of motion and use by bilateral amputees. This technique, however, has many downsides. Because every person has unique EMG signals, a unique algorithm decoding those signals must be developed for every user. Developing a personal algorithm for every user is expensive and requires a significant investment of time. Moreover, therapy may be inefficient or fruitless if a user cannot consistently control their EMG signals, which can be the case with some amputees. Failure after such a prolonged effort to develop an algorithm risks hindering an amputee's motivation and exacerbating the already prevalent issue of therapy patients not following through and completing therapy.

Sensor-based techniques may offer a reliable and economical approach to phantom limb pain therapy. Sensors and/or cameras may be used to track the movements of intact portions of an amputee's body. The tracked movements may then be used to provide a representation of a phantom limb as a simulated full limb to an amputee. In some approaches, sensors track the movement of an intact limb and provide a mirror image copy of that movement for a simulated full limb. However, such representations are limited to synchronous movements, which limits engagement. In other approaches, movements of partially intact limbs may used to generate representations of a complete limb. These techniques may face the challenge of animating limbs having multiple joints from limited tracking data. For instance, some approaches may track shin position and animate a visual representation of a complete leg. However, tracking data indicating shin position can often fail to provide information regarding foot position that may vary, e.g., according to ankle flexion, which may result in a disjointed or clunky animation. Similar issues may arise when relying on tracking data of an upper arm or forearm alone to, e.g., animate an arm with a hand. The present disclosure provides solutions for rendering and animation that may address some of these shortcomings.

Phantom limb pain therapy may present many challenges. As a general baseline, any effective technique should provide sensory feedback that accurately matches an amputee's expectations. One of phantom limb therapy's key objectives is to help establish a match between visual expectations and sensory feedback, e.g., to help put the mind at ease. Such therapy attempts to normalize the cortical representation of the missing limb and improve the correspondence between actual and predicted sensory feedback. One goal may be to provide multisensory feedback to facilitate neuroplasticity.

A further challenge may be to enhance amputee engagement with therapy. Traditional therapy may not be very fun for many people, and this is evidenced by the fact that many therapy patients never fully complete their prescribed therapy regime. There exists a need to make therapy more enjoyable. One possible avenue is to provide an immersive experience, which virtual reality is particularly well posed to provide.

### Virtual Reality Therapy

Virtual reality can offer a therapy experience that is immersive in many ways. Generally, VR systems can be used to instruct users in their movements while therapeutic VR can replicate practical exercises that may promote rehabilitative goals such as physical development and neurorehabilitation in a safe, supervised environment. For instance, patients may use physical therapy for treatment to improve coordination and mobility. Physical therapy, and occupational therapy, may help patients with movement disorders develop physically and mentally to better perform everyday living functions.

A VR system may use an avatar of the patient and animate the avatar in the virtual world. VR systems can depict avatars performing actions that a patient with physical and/or neurological disorders may not be able to fully execute. A VR environment can be visually immersive and engross a user with numerous interesting things to look at. Virtual reality therapy can provide (1) tactile immersion with activities that require action, focus, and skill, (2) strategic immersion with activities that require focused thinking and problem solving, and (3) narrative immersion with stories that maintain attention and invoke the imagination. The more immersive the environment is, the more a user can be present in the environment. Such an engrossing environment allows users to suspend disbelief in the virtual environment and allow users to feel physically present in the virtual world. While an immersive and engrossing virtual environment holds an amputee's attention during therapy, it is activities that provide replay value, challenges, engagement, feedback, progress tracking, achievements, and other similar features that encourage a user to come back for follow up therapy sessions.

Using sensors in VR implementations of therapy allows for real-world data collection as the sensors can capture movements of body parts such as hands, arms, head, neck, back, and trunk, as well as legs and feet in some instances, a system to convert and animate an avatar in a virtual environment. Such an approach may approximate the real-world movements of a patient to a high degree of accuracy in virtual-world movements. Data from the many sensors may be able to produce visual and statistical feedback for viewing and analysis by doctors and therapists. Generally, a VR system collects raw sensor data from patient movements, filters the raw data, passes the filtered data to an inverse kinematics (IK) engine, and then an avatar solver may generate a skeleton and mesh in order to render the patient's avatar. Typically, avatar animations in a virtual world may closely mimic the real-world movements, but virtual movements may be exaggerated and/or modified in order to aid in therapeutic activities. Visualization of patient movements through avatar animation could stimulate and promote recovery. Visualization of patient movements may also be vital for therapists observing in person or virtually.

A VR environment rendering engine on an HMD (sometimes referred to herein as a “VR application”), such as the Unreal® Engine, may use the position and orientation data to generate a virtual world including an avatar that mimics the patient's movement and view. Unreal Engine is a software-development environment with a suite of developer tools designed for developers to build real-time 3D video games, virtual and augmented reality graphics, immersive technology simulations, 3D videos, digital interface platforms, and other computer-generated graphics and worlds. A VR application may incorporate the Unreal Engine or another three-dimensional environment developing platform, e.g., sometimes referred to as a VR engine or a game engine. Some embodiments may utilize a VR application, stored and executed by one or more of the processors and memory of a headset, server, tablet and/or other device to render a virtual world and avatar. For instance, a VR application may be incorporated in one or more of head-mounted display 201 and clinician tablet 210 of FIGS. 10A-D and/or the systems of FIGS. 12-13.

Some embodiments may utilize a VR engine, e.g., as part of a VR application, stored and executed by one or more of the processors and memory of a headset, server, tablet and/or other device. For instance, VR engine may be incorporated in one or more of head-mounted display 201 and clinician tablet 210 of FIGS. 10A-D and/or the systems of FIGS. 12-13. A VR engine may run on a component of a tablet, HMD, server, display, television, set-top box, computer, smart phone, or other device.

### Challenges in VR Amputee Therapy

A particularly challenging aspect of the immersive process is the generation of a sense of unity between a user and an avatar that represents them. An avatar should look like a user (at least generally) and it should move according to the patient's volition. A user should feel a sense of control over an avatar and this control must have high fidelity to convincingly establish a sense of unity. This is important not only for the body parts that are tracked and represented to a user, but also the representation of a user's missing limb(s), e.g., simulated full limb(s). Throughout the present disclosure, a missing limb in the real world that is rendered and animated as part of a virtual avatar may be referred to as a “virtual simulated full limb,” “simulated full limb,” “virtual full limb,” and the like. The fidelity of control over virtual simulated full limbs approaches the level of control over one's tracked, intact limbs. A system may be immersive if it can establish an illusion that the virtual avatar is real and trick a user's brain into believing a simulated full limb is an extension of their own body and under their volitional control.

Fidelity of control over simulated full limbs may be one of the most challenging aspects of avatar rendering. In order to render a convincing virtual simulated full limb, it is necessary that the predictive strength of the VR therapy system be very strong to accurately predict what movements a user intends for their virtual simulated full limbs. Additionally, such accuracy must be consistently performed as momentary breaks in the fidelity of movement risks shattering the suspension of disbelief in the virtual world. For instance, a jerky or unsmooth motion of a virtual simulated full limb may offer an unwelcome moment of clarity that reminds a user that what they see is actually virtual, thereby causing their minds rise above the immersion. Inconsistency of animation could hamper engagement in VR therapy.

Maintaining immersion and suspension of disbelief in the virtual world may also require freedom to perform a variety of movements. A user cannot feel a believable sense of control over their virtual simulated full limb if they cannot instruct it to do what he or she desires. Limiting the movements of a user, reduces the immersive potential of the experience. As such, a primary challenge is in developing activities that permit a user to perform a variety of movements, while still providing movement visualizations for a virtual simulated full limb that match expectations. One of the major downsides of mirror-based therapy (e.g., with mirrors, robotics, or some kinds of virtual therapy) is that it is restricted to only mirrored synchronous movements. Ideally, new methods of therapy enable activities that allow other types of movements. Using VR animation techniques, there's a possibility of a greater variety of movements beneficially increases a user's sense of control over their virtual simulated full limb. Thus, there exists a need to increase the variety of activities and movements that a user can perform during amputee therapy that may achieve a therapeutic effect.

A further challenge is to animate movements in an avatar in real time based on received tracking data and predicted movements for a virtual simulation of a regenerated limb, e.g., updating avatar position at a frequency of 60 times per second (or higher). In some animation approaches, animators may have the luxury of animating movements far in advance for later display, or at least ample time to develop a workable, predefined set of allowable movements. This is not the case here when, e.g., a VR system is animating an avatar based on a user's tracked movements. It is not reasonably feasible to animate every possible movement in advance and limiting the range of motions allowed by a user risks ruining immersion and/or hurting engagement. Instead, animated movements must be based on a hierarchy of rules and protocols. As such, teachings of animation techniques not based on tracking data bear little relevance to the complex methods of animating an avatar in real time, e.g., based on live tracking data.

Avatar animations based on tracking data are generated according a series of predefined rules, such as forward kinematics, inverse kinematics, forwards and backwards reaching inverse kinematics, key pose matching and blending, and related methods. These rules and models of human kinematics enable rendering in real time and accommodate nearly limitless input commands, which allows a 3D model to be deformed into any position a person could bend themselves into. These rules and models of human movement offer a real-time rendering solution beyond traditional animation methods.

The challenges of live rendering may be further exacerbated by tasking a VR system to predict and determine movements made by untracked limbs/body parts using only the current and past position of tracked limbs and rules and models of movement. For instance, a system is challenged to animate a virtual simulated full limb that moves accurately and predictably without any tracking data for that virtual simulated full limb because there is nothing to track. Animating avatars in real time The rules and models that drive the animations of untracked limbs and body parts when is an emerging art.

The live rendering pipeline typically consists of collecting tracking data from sensors, cameras, or some combination thereof. Sensor data and tracking data may be referred to interchangeably in this disclosure. Tracking data may then be used to generate or deform a 3D model into positions and orientations provided by tracking data. The 3D model is typically comprised of a skeletal hierarchy that enables inherited movements and a mesh that provides a surface topology for visual representation. The skeletal hierarchy is comprised of a series of bones where every bone has at least one parent, wherein movements of parent bones influence movements of each child bone. Generally, movements of a parent bone cannot directly determine movements of an articulating joint downstream, e.g., the movements of one of its children. For example, movements of an upper arm or a forearm cannot directly provide information on what wrist flexion should be animated. To animate body parts on either side of an articulating joint, typically tracking data must be acquired for at least a child connecting to the joint. Kinematics models can determine body position if every joint angle is known and provided (e.g., forward kinematics), or if the position of the last link in the model (e.g., an end effector or terminal child bone) is provided (e.g., inverse kinematics or FABRIK). However, standard models cannot accommodate both a lack of every joint angle and a lack of an end effector position and orientation, and this is what is needed to animate a virtual simulated full limb for an amputee. There is either no tracking data for the missing limb or only partial tracking data for the missing limb. Even if some tracking data is available, it does not provide an end effector and it cannot provide every joint angle. Thus, the standard kinematic models do not have the information they need to model kinematic data. What is needed is a new kinematic model that can utilize available tracking data to predict and determine movements of an end effector for which tracking data is categorically unavailable. The more joints there are beyond the available tracking data the more difficult it is to predict. The higher the ratio of predicted tracking data to available tracking data the more difficult it is to predict.

### Simulated Full Limbs in VR Amputee Therapy

The present disclosure details a virtual reality system that displays to a user having an amputated limb a virtual simulated full limb that is believable and easily controlled. The present disclosure also details a method for animating a simulated full limb that appears to move under a user's volition by using rules, symmetries, props, specific activities, or some combination thereof. In one particular example, an embodiment may use a modified method of inverse kinematics that artificially and arbitrarily overrides an end effector of a limb and thereby provides animations that are believable and easily controlled.

The present disclosure may offer an animation solution that generates predictable and controllable movements for an amputee's virtual simulated full limb. Some embodiments may establish a match between expected and visualized movements that help alleviate virtual simulated full limb pain. The technique benefits from requiring minimal setup and from being economical. Some embodiments may come packaged with games and activities that provide engagement, immersion, and replay value to enhance the rehab experience and help facilitate rehab completion. Additionally, some embodiments may include activities that permit a variety of different movement options, while still providing animations and visualizations that meet expectations.

In some embodiments, virtual simulated full limb pain therapy may be conducted via a virtual reality system. For example, a user wears a head mounted display (“HMD”) that provides access to a virtual world. The virtual world provides various immersive activities. A user interacts with the virtual world using an avatar that represents them. One or more methods may be utilized to track a user's movements and an avatar is animated making those same, tracked movements. In some embodiments, an avatar is full bodied, and the tracked movements of a user inform or determine the movements that are animated for the missing limb, e.g., the “simulated full limb” or “virtual simulated full limb.”

The movements of a user may be tracked with one or more sensors, cameras, or both. In one example, a user is fitted with one or more electromagnetic wearable sensors that wirelessly collect and report position and orientation data to an integrated computing environment. The computing environment collects and processes the position and orientation data from each source of tracking data. The tracking data may be used to generate or deform a 3D model of a person. A first set of received tracking data may be used to generate a 3D model having the same position and orientations as reported by the sensors. With each subsequent set of tracking data, the portions of the 3D model for which updated tracking data is received may be deformed to the new tracked positions and orientations. The 3D model may be comprised of a skeletal structure, with numerous bones having either a parent or child relationship with each attached bone, and a skin that represents a surface topology of the 3D model. The skin may be rendered for visual display as an avatar, e.g., an animation of an avatar. The skeletal structure preferably enables complete deformation of the 3D model when tracking data is only collected for a portion of the 3D model by using a set of rules or parameters.

In one example, a user is fitted with one more wearable sensors to track movements and report position and orientation data. A user may have an amputated limb and an intact limb. A sensor may be placed at or near an end of the intact limb, at or near the end of the amputated limb (e.g., a “stump”), or both. Sensors may be placed on a prosthetic limb and/or end effector. Sensors may be uniform and attachable to any body part, may be specialized to attach to specific body parts, or some combination thereof. Uniform sensors may be manually assigned to specific body locations or the sensors may automatically determine where on the body they are positioned. The sensors may track movements and report position and orientation data to a computing environment.

A computing environment may be used to process sensor data. The computing environment may map the tracking data onto a 3D model. The tracking data may be mapped onto the 3D model by deforming the 3D model into the positions and orientations reported by the sensors. For instance, a sensor may track a user's right hand at a given position and orientation relative to a user's torso. The computing environment maps this tracking data onto the 3D model by deforming the right hand of the 3D model to match the position and orientation reported by the sensor. After the tracking data has been mapped to the 3D model, one or more kinematic models may be employed to determine the position of the rest of the 3D model. Once the 3D model is fully repositioned based on the tracking data and the kinematic models, a rendering of the surface topology of the 3D model may be provided for display as an avatar.

Tracking data is only available for those body parts where sensors are placed or those body parts that are positioned within line of sight of a camera, which of course may vary with movement. Portions of the body without tracking data represent gaps in the tracking data. Some gaps in tracking data may be solved by traditional animations methods, such as inverse kinematics. However, traditionally, inverse kinematics relies on a known position and orientation data for an end effector, and such data is categorically unavailable for the animation of a simulated full limb. For example, tracking data for a hand often functions as an end effector for an arm. If an amputee is missing a hand, then the traditional end effector is categorically unavailable and animations of such a hand must rely on non-traditional animations techniques, such as those disclosed herein.

In some embodiments, a modified inverse kinematics method is used to solve the position of a full-bodied 3D model based on tracking data collected from an amputee. Tracking data is preferably collected from the amputated limb's fully intact partner limb. At least a portion of the tracking data should correspond to a location that is at or near the end of the fully intact limb. This tracking data may be assigned as an end effector for the fully intact limb. Using this tracking data as an end effector, an end of a limb of the 3D model can be deformed to the position and orientation reported by the tracking data and then inverse kinematics can solve the parent bones of that end effector to deform the entire limb to match the tracked end effector. Inverse kinematics may further use joint target locations or pole vectors to assist in realistic limb movements. Additionally, the modified inverse kinematics method solves not only the tracked fully intact limb, but also solves a position of a virtual simulated full limb. The modified inverse kinematics method may be executed in a number of ways, as elaborated in more detail in what follows, to establish an end effector for a virtual simulated full limb that moves, or at least appears to move, under the volition of a user. In some instances, the inverse kinematics method may access a key pose library comprised of predefined positions and may use such key poses or blends thereof to render surface topology animations of an avatar.

### Relational Movements of Limbs in VR Amputee Therapy

Some embodiments may provide virtual simulated full limb animations that are informed by available data, e.g., tracking data and/or sensor data. A virtual simulated full limb's position, orientation, and movements may be informed by available tracking data. Tracking data from an intact partner of a missing limb may inform the animations displayed for a virtual simulated full limb. Additionally, tracking data from the rest of a user's body may inform the animations displayed for a virtual simulated full limb. In one example, tracking data collected from a stump informs the animations displayed for a virtual simulated full limb. In another example, tracking data collected from a head, a shoulder, a chest, a waist, an elbow, a hip, a knee, another limb, or some combination thereof may inform the animations displayed for a simulated full limb. Virtual simulated full limb animations may also be informed by the particular activity or type of activity that is being performed. A virtual activity may require two limbs to move in a particular orientation relative to one another, in a particular pattern, or in relation to one or more interactive virtual objects. Any expected or required movements of an activity may inform the animations displayed for a virtual simulated full limb.

Virtual simulated full limb animations may be determined, predicted, or modulated using a relation between a virtual simulated full limb and the tracking data that is received. The relation may establish boundaries between the movements of an intact limb and the corresponding virtual simulated full limb, e.g., two partner limbs. Boundaries between two limbs or two body parts may be established by a bounding box or accessed constraint that interconnects relative movement of the two limbs or two body parts. A relation may operate according to a set of rules that translates tracked limb movements into corresponding or synchronizing virtual simulated full limb movements. The relation may establish an alignment or correlation between partner limb movements or between two body parts. The relation may establish a symmetry between partner limbs, such as a mirrored symmetry. The relation may establish a tether between two partner limbs or two body parts, whereby their movements are intertwined. The relation may fix a virtual simulated full limb relative to a position, orientation, or movement of a tracked limb or body part.

A virtual simulated full limb's position, orientation, and movements may be aligned or correlated with tracking data. In one example, a virtual simulated full limb is aligned with the tracked movements of its intact partner limb. A virtual simulated full limb may be aligned with its partner limb across one more axes of a coordinate plane. Alignment may be dictated by the activity being performed or any objects or props that are used during the activity. In another example, a virtual simulated full limb is correlated with the tracked movements of its partner limb. A virtual simulated full limb may be affected by the position, orientation, and movements of its partner limb. Alternatively, a virtual simulated full limb may be connected with or depend upon its partner limb and its tracked position, orientation, and movements. In some instances, movements of a virtual simulated full limb may lag behind the movement of a tracked limb. Additionally, movements of a tracked limb may need to reach a minimum or maximum threshold before they are translated into corresponding virtual simulated full limb movements. The minimum or maximum threshold may rely on relative distance, rotation, or some combination of distance and rotation that is either set or variable.

A virtual simulated full limb's position, orientation, and movements may be determined by tracking data. In one example, a position, orientation, and movement of a virtual simulated full limb are determined by a partner limb's position, orientation, and movement. For instance, movements of a partner limb along an X, Y, or Z axis may determine corresponding movements or rotations for a virtual simulated full limb. Rotations of a partner limb may determine movements or rotations for a virtual simulated full limb. The movements of a tracked limb may determine virtual simulated full limb movements according to a pre-defined set of rules correlating motion between the two limbs. Additionally, a virtual simulated full limb's display may be at least partially determined by, e.g., an activity or a prop or object used during an activity.

A simulated full limb may be displayed in symmetry with tracking data. In one example, a position, orientation, movement, or some combination thereof of a virtual simulated full limb are symmetrical to at least some portion of a partner limb's position, orientation, and movement. Movements between two partner limbs may be parallel, opposite, or mirrored. The symmetry between partner limbs may depend on an axis traversed by the movement. Rotations of a tracked limb may be translated into symmetrical movements or rotations of a virtual simulated full limb. In one example, a rotation of a tracked limb results in geosynchronous movements of a virtual simulated full limb.

Some embodiments may, for example, animate an avatar performing an activity in virtual reality by receiving sensor data comprising position and orientation data for a plurality of body parts, generating avatar skeletal data based on the position and orientation data, and identifying a missing limb in the first skeletal data. The system may access a set of movement rules corresponding to the activity. For example, movement rules may comprise symmetry rules, predefined position rules, and/or prop position rules. The system may generate virtual simulated full limb data based on the set of movement rules and the avatar skeletal data and render the avatar skeletal data with simulated full limb skeletal data. In some embodiments, generating virtual simulated full limb data may be based on the set of movement rules and avatar skeletal data comprising, e.g., a relational position for a full limb. In some embodiments accessing the set of movement rules corresponding to the activity comprises determining a movement pattern associated with the activity and accessing the set of movement rules corresponding to the movement pattern.

Some embodiments may be applicable to patients with other physical and neurological impairments separate or in addition to one or more amputated limbs. For instance, some or all embodiments may be applicable to patients who may have experienced paralysis, palsy, strokes, nerve damage, tremors, and other brain or body injuries.

### BRIEF DESCRIPTION OF THE DRAWINGS

FIG. 1 is an illustrative depiction of a virtual mirror for generating mirrored data from tracking data, in accordance with some embodiments of the disclosure;

FIG. 2A is an illustrative depiction of a rule-based symmetry between a tracked limb and a virtual simulated full limb, in accordance with some embodiments of the disclosure;

FIG. 2B is an illustrative depiction of a rule-based symmetry between a tracked limb and a virtual simulated full limb, in accordance with some embodiments of the disclosure;

FIG. 2C is an illustrative depiction of a rule-based symmetry between a tracked limb and a virtual simulated full limb, in accordance with some embodiments of the disclosure;

FIG. 2D is an illustrative depiction of a rule-based symmetry between a tracked limb and a virtual simulated full limb, in accordance with some embodiments of the disclosure;

FIG. 3 is an illustrative depiction of a virtual reality driving activity, in accordance with some embodiments of the disclosure;

FIG. 4 is an illustrative depiction of a virtual reality baseball activity, in accordance with some embodiments of the disclosure;

FIG. 5 is an illustrative depiction of a virtual reality bicycle riding activity, in accordance with some embodiments of the disclosure;

FIG. 6 is an illustrative depiction of a virtual reality kayaking activity, in accordance with some embodiments of the disclosure;

FIG. 7 is an illustrative depiction of a virtual reality towel wringing activity, in accordance with some embodiments of the disclosure;

FIG. 8 is an illustrative depiction of a virtual reality accordion playing activity, in accordance with some embodiments of the disclosure;

FIG. 9 depicts an illustrative flow chart of a process for overriding position and orientation data with a simulated full limb, in accordance with some embodiments of the disclosure;

FIG. 10A is a diagram of an illustrative system, in accordance with some embodiments of the disclosure;

FIG. 10B is a diagram of an illustrative system, in accordance with some embodiments of the disclosure;

FIG. 10C is a diagram of an illustrative system, in accordance with some embodiments of the disclosure;

FIG. 10D is a diagram of an illustrative system, in accordance with some embodiments of the disclosure;

FIG. 11A is a diagram of an illustrative system, in accordance with some embodiments of the disclosure;

FIG. 11B is a diagram of an illustrative system, in accordance with some embodiments of the disclosure;

FIG. 11C is a diagram of an illustrative system, in accordance with some embodiments of the disclosure;

FIG. 12 is a diagram of an illustrative system, in accordance with some embodiments of the disclosure; and

FIG. 13 is a diagram of an illustrative system, in accordance with some embodiments of the disclosure.

### DETAILED DESCRIPTION

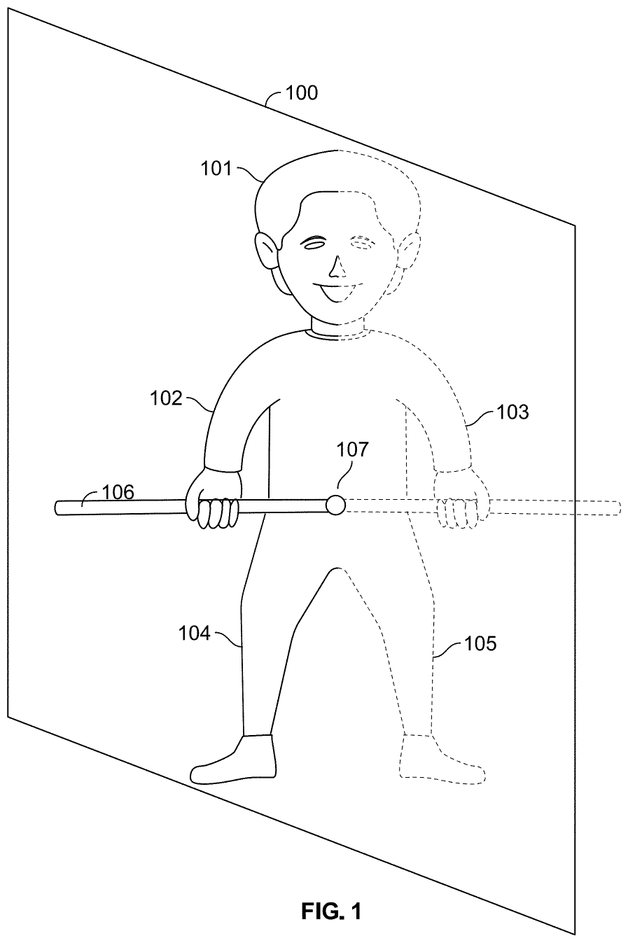

FIG. 1 is an illustrative depiction of a virtual mirror for generating mirrored data from tracking data, in accordance with some embodiments of the disclosure. For instance, FIG. 1 illustrates an example of a virtual mirror 100 that may be used to generate animations. The virtual mirror may generate mirrored copies of a user's tracked movements as mirrored data. Mirrored data and tracking data may be combined to deform a 3D model and a surface topology of the 3D model may be rendered for display as an avatar 101. In one example, the movements of a tracked limb may determine the animations generated for both that limb and its partner limb. Tracking data may directly determine a position and orientation of a limb and mirrored data may indirectly determine the position and orientation of a partner limb. For instance, the VR engine may receive tracking data for a position and orientation of a tracked arm 102 that the virtual mirror 100 may generate mirrored data of that determines a position and orientation for a virtual simulated full arm 103. Similarly, the VR engine may receive tracking data for a tracked leg 104 that the virtual mirror may copy to generate mirrored data for a virtual simulated full leg 105. The virtual mirror 100 may be especially useful for generating animations of virtual simulated full limbs when a user performs a virtual reality activity that requires synchronized limb movements.

The virtual mirror 100 of FIG. 1 may also be useful for rendering animations when tracking data for one limb is complete and tracking data for a partner limb is partial or incomplete. This functionality may be especially useful when a limb of a user is partially amputated and tracking data is received from the intact stump, e.g., an elbow stump of an arm. In one example, the VR engine may receive tracking data for an elbow and a hand of a first arm and tracking data for only an elbow of a second arm. With tracking data on both sides of the virtual mirror, the VR engine may generate mirrored data that is duplicative of tracked data. In this example, the mirror may generate mirrored data for an elbow position and orientation of the first arm, which is duplicative of the complete tracking data for the first arm, and mirrored data for a complete second arm position and orientation, which is duplication of the elbow tracking data for the second arm.

Mirrored data that is duplicative may be used to inform the animations that are rendered. For instance, duplicative mirrored data may be combined with tracked data according to a weighting system and the resulting combination, e.g., mixed data, is used to deform a 3D model that forms the basis of a rendered display. Mixed data results from weighted averages of tracked data and mirrored data for the same body part, adjacent body parts, or some combination thereof. The mixed data may be weighted evenly as 50% tracked data and 50% mirrored data. Alternatively, the weighting can be anywhere between 0-100% for either the tracked data or the mirrored data, with the remaining balance assigned to the other data set. This weighting system remedies issues that could arise if, for example, the tracked position of an elbow of a user's amputated arm did not align with the mirrored data for a forearm sourced from a user's intact partner arm. Rather than display an arm that is disconnected or inappropriately attached, the weighting system generates an intact and properly configured arm that is positioned according to a weighted combination of the tracking data and the mirrored data. This process may be facilitated by a 3D model, onto which tracked data, mirrored data, and mixed data are mapped, that is restricted by a skeletal structure that only allows anatomically correct position and orientations for each limb and body part. Any position and orientation data that would position or orient the 3D model into an anatomically incorrect position may be categorically excluded or blended with other data until an anatomically correct position is achieved.

The manner in which duplicative data is compiled may vary with the activity a user is performing in virtual reality. During some activities, the VR engine may preferentially render for display one set of duplicative data over the other set rather than using a weighted average. In one example, the VR engine may use an alignment tool to determine how to parse duplicative data. For instance, the VR engine may receive tracking data for a first arm and tracking data for an elbow of a second arm, the virtual mirror may generate mirrored data for an elbow position and orientation of the first arm and mirrored data for a position and orientation of the second arm, and utilize an alignment tool to determine which set of duplicative data is used to render an avatar 101. The alignment tool may come in the form of a prop 106 that is held by two hands. In this example, a user may be physically gripping the prop 106 with their first arm, e.g., tracked arm 102. With this alignment tool, the VR engine may preferentially render an avatar with tracking data for the first arm and mirrored data for the second arm, e.g., virtual simulated full limb 103. The VR engine may disregard tracking data from the elbow of the second arm that would position the second arm such that it could not grip a virtual rendering of the prop 106 and may also disregard mirrored data for the first arm 102 that would do the same. This preferential rendering is especially useful when a user is performing an activity where they contact or grip an object.

Although previous examples have focused on the generation of mirrored data for limbs and the parsing between duplicative data for two limbs for simplicities sake, it should be understood that the mirror may generate mirrored data for any body part for which tracking data is received. For instance, tracking data for the position and orientation of shoulders, torsos, and hips may be utilized by the virtual mirror 100 to generate mirrored data of those body parts. Alternatively, the virtual mirror 100 may be configured to only establish a symmetry between two specific portions, regions, or sections of a user. The virtual mirror 100 may only generate mirrored data for a specific limb, while not providing mirrored copies of any other body part. For example, the virtual mirror 100 may establish a symmetry between the two limbs, such that the position and orientation of one is always mirrored by its partner's position and orientation, while the remainder of an avatar is positioned from tracking data without the assistance of the virtual mirror 100.

The nature of the mirrored copies depends on the position and orientation of the virtual mirror 100. In the example illustrated by FIG. 1, the virtual mirror 100 is positioned at a midline of an avatar 101. The position and orientation of the virtual mirror 100 may be stationary or it may translate according to a user's tracked movements. For instance, to consistently mirror body parts having the ability to move with many degrees of freedom, the virtual mirror may have a dynamic position and orientation that adjusts according to the position of one or more tracked body parts.

A virtual mirror 100 that translates may translate across a pivot point 107, may translate across one or more axes of movement, or some combination thereof. In one example, the position and orientation of the virtual mirror 100 is controlled by a prop 106. As a user is tracked as moving the prop 106, the virtual mirror 100 moves as if it is attached the virtual mirror 100 at the pivot point 107. The prop 106 may fix the distance between two arms and the prop may fix the virtual mirror 100 at a set distance from the tracked limb that is adhered to the prop. In some embodiments, a prop may not be used, and the position and orientation of the mirror may depend on a tracked limb directly. In one example, a mirror is positioned at a center point, e.g., pivot point 107, that aligns with a midline of an avatar 101. If a limb is tracked as crossing the midline, the mirror may flip and animate a limb as crossing. The height of the pivot point may be at a mean between the heights of a user's limbs. The angle of the tracked limb may determine the relative orientation of a limbs as they cross, e.g., one on top of the other. In some instances, the mirrored data may be repositioned according to the orientation of the tracked limb. For instance, if tracking data for an arm indicates that the thumb is pointing upwards and the arm is crossing the chest, then the mirrored data for a virtual simulated full limb may be positioned such that it is above the tracked arm and shows no overlap. Likewise, if the thumb is pointed down, the mirrored data will be adjusted vertically and the angle adjust accordingly, such that a simulated full limb is positioned beneath the tracked arm. In some instances, the VR engine may not only utilize tracking data to generate mirrored data but may also simply copy one or more features of the tracked limbs position or movement. In such cases, the VR engine may generate parallel data in addition to mirrored data, and an avatar may be rendered according to some combination of tracked data, mirrored data, and parallel data along with anatomical adjustments that prevent unrealistic overlap, position, or orientation.

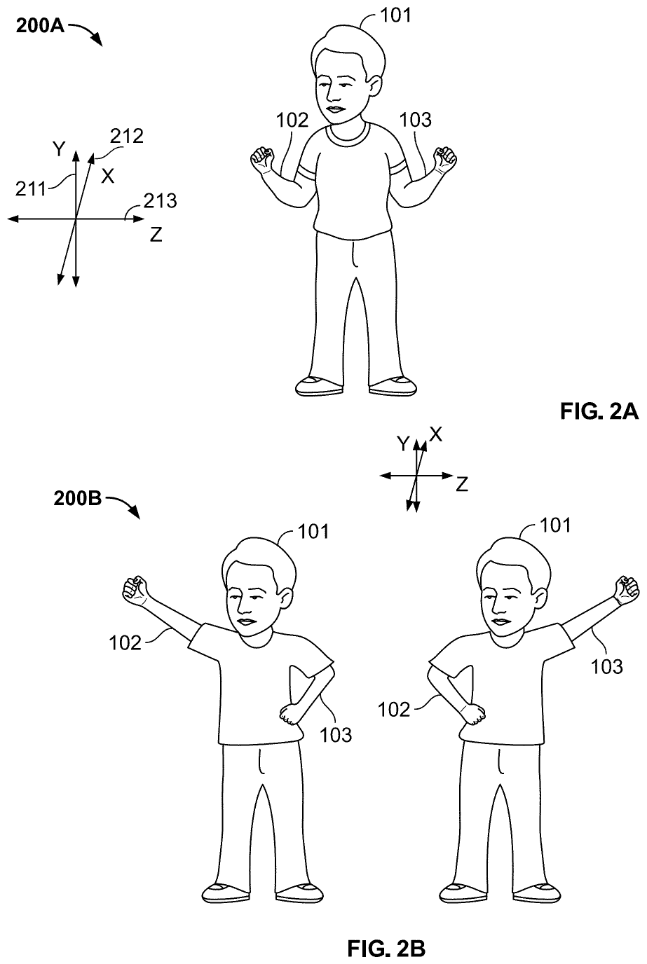

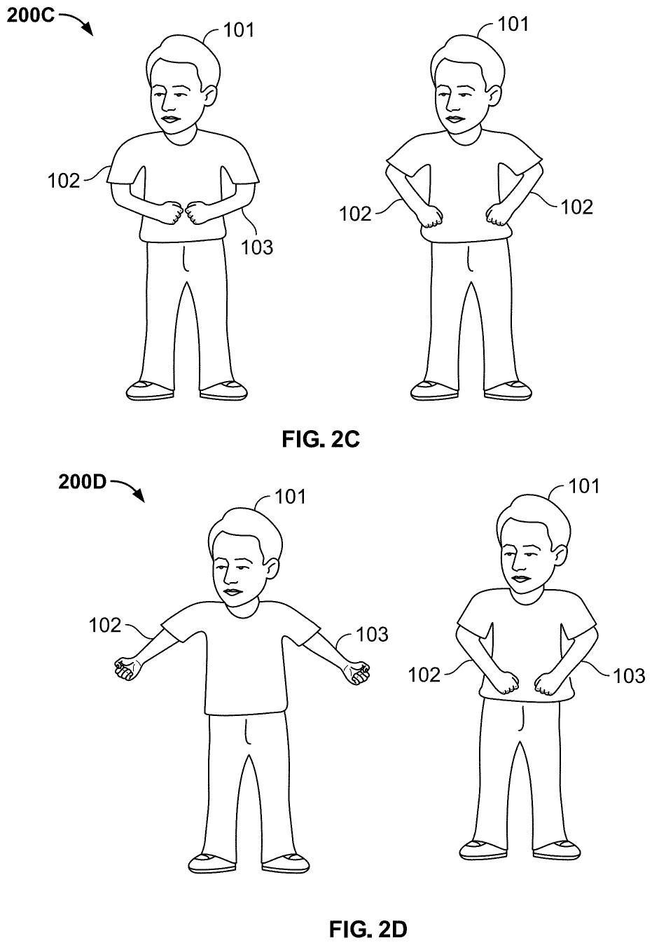

FIGS. 2A-D are illustrative depictions of rule-based symmetry between a tracked limb and a virtual simulated full limb, in accordance with some embodiments of the disclosure. For instance, FIGS. 2A-D illustrate examples of a rule-based symmetry that may executed between a tracked arm 102 and a virtual simulated full arm 103 of an avatar 101. The VR engine may receive tracking data for a tracked arm 102 that may be tracked as moving along any axes 200A. For instance, a tracked arm or leg may move along the Y-axis 211, the X-axis 212, the Z-axis 213, or some combination thereof. For simplicities sake, an avatar 101 in these examples is positioned with shoulders along the Z-axis 213. From an avatar's 101 perspective in this position, the arms move up and down along the Y-axis 211, they move forwards and backwards along the X-axis 212, and they move left and right along the Z-axis 213. The rule-based symmetry utilizes the tracking data received for the tracked arm 102 to determine what movements are rendered for display by a virtual simulated full arm 103. In some examples, movements along a certain axis may be parallel, opposite, mirrored, or rotationally connected. Such rules may be static or variable, may vary from one activity to another, and may simply vary depending on the manner in which the movement is described.

FIG. 2A is an illustrative depiction of a rule-based symmetry between a tracked limb and a virtual simulated full limb, in accordance with some embodiments of the disclosure. For instance, FIG. 2A illustrates an example of an avatar 101 having a tracked arm 102 positioned outstretched and directly in front of an avatar 101. This position may be referred to as a set or fixed as a neutral position or starting position for explanatory ease. When the VR engine receives tracking data indicating that the tracked arm 102 is in this position, the VR engine may generate position and orientation data for a virtual simulated full arm 103 such that it will occupy a mirrored position of the tracked arm 102. In some instances, the rule-based symmetry may generate parallel, opposite, mirrored, or rotationally connected data for a position, an orientation, or some combination thereof of tracking data and render a selection of that data, a portion of that data, or a combination of that data for display.

FIG. 2B is an illustrative depiction of a rule-based symmetry between a tracked limb and a virtual simulated full limb, in accordance with some embodiments of the disclosure. For instance, FIG. 2B illustrates an example of an opposite movement pattern 200B where the tracked arm 102 has moved up and down along the Y-axis 211 relative to the neutral position illustrated in FIG. 2A. In this example, the rule based symmetry applies an opposite symmetry for limbs moving along the Y-axis 211, such that tracking data indicating that the tracked arm 102 is positioned upwards along the Y-axis 211 is utilized by the VR engine to generate opposite data, such that a virtual simulated full arm 103 is rendered in a position that is downwards on the Y-axis 211. Likewise, when tracking data is received indicating that the tracked arm 102 is down along the Y-axis 211 the VR engine will generate a position for a virtual simulated full arm 103 that is orientated upwards in an opposite fashion.

Renderings of opposite movements of a tracked limb may be useful for rendering animations for user a performing a synchronized activity or an activity having synchronized control mechanisms. Although the positions are rendered as opposite along the Y-axis 211 in this example, the rotational orientation of the tracked arm 102 may be used to generate a rotational orientation of a virtual simulated full limb that is either mirrored or parallel. For instance, the palms of both arms may be rendered as facing towards the body in a mirrored fashion. Alternatively, the palms of the arms may be pointing in the same direction in a parallel fashion. The manner in which rotational orientation of a tracked limb 102 is used to determine rotational orientation of a virtual simulated full arm 103 may vary from one activity to another.

FIG. 2C is an illustrative depiction of a rule-based symmetry between a tracked limb and a simulated full limb, in accordance with some embodiments of the disclosure. For instance, FIG. 2C illustrates an example of a parallel movement pattern 200C where the tracked arm 102 has moved inwards and outwards along the X-axis 212 relative to the neutral position illustrated in FIG. 2A. In this example, the rule based symmetry applies a parallel symmetry for limbs moving along the X-axis 212, such that tracking data indicating the tracked arm 102 is positioned inwards along the X-axis 212 is utilized by the VR engine to generate parallel data, such that a simulated full arm 103 is rendered in a position that is inwards along the X-axis 212. Likewise, when tracking data is received indicating that the tracked arm 102 is outwards along the X-axis 212 the VR engine will generate a position for a simulated full arm 103 that is orientated outwards in a parallel fashion. Although the relative movements of the tracked arm 102 and a simulated full arm 103 are parallel along the X-axis in this example, the relative rotational orientation may be static or variable and may consist of a mirrored relative orientation, a parallel orientation, or some combination thereof.

FIG. 2D is an illustrative depiction of a rule-based symmetry between a tracked limb and a simulated full limb, in accordance with some embodiments of the disclosure. For instance, FIG. 2D illustrates another example of a parallel movement pattern 200D where the tracked arm 102 has moved towards the midline (e.g., the right arm moving to the left) and outwards from the midline (e.g., the right arm moving to the right) along the Z-axis 213 relative to the neutral position illustrated in FIG. 2A. In this example, the rule based symmetry applies a parallel symmetry for limbs moving along the Z-axis 213, such that tracking data indicating the tracked arm 102 is positioned outwards from the midline along the Z-axis 213 is utilized by the VR engine to generate parallel data, such that a simulated full arm 103 is rendered in a position that is also positioned outwards from the midline along the X-axis 212. Likewise, when tracking data is received indicating that the tracked arm 102 has moved towards the midline along the Z-axis 213 the VR engine will generate a position for a simulated full arm 103 that is orientated towards the midline in a parallel fashion. Although the relative movements of the tracked arm 102 and a simulated full arm 103 are parallel along the Z-axis 213 in this example, the relative rotational orientation may be static or variable and may consist of a mirrored relative orientation, a parallel orientation, or some combination thereof.

In the examples illustrated by FIGS. 2A-D, rule-based symmetry was either parallel or opposite, e.g., depending on the axis of movement of the tracked arm 102. However, natural movement typically entails moving arms along more than one such axis. In such instances, the rule-based symmetry may apply a symmetry that is weighted between a parallel and opposite position. For instance, the VR engine may receive tracking data for a tracked arm 102 that indicates that the arm has moved, relative to the neutral position, up along the Y-axis 211 and away from the midline along the Z-axis. In such instances, the VR engine may generate a position of a simulated full arm 103 having an orientation opposite along the Y-axis and parallel along the Z-axis. Similar combinations may be made across different axes of movements and in some instances weighted averages may be used to increase the influence of one rule-based symmetry over another. In some embodiments, rules of motion between the axes 200A described herein may be varied without departing from the scope of the present disclosure. Once a user learns the rules of movement of a given activity, the rules beneficially allow a user to know with confidence what movements their simulated full limb is going to make based on the movements that he or she makes with their tracked limb. This provides the much-needed match between sensory and tactile feedback that can help alleviate simulated full limb pain.

### End Effector Overridden Inverse Kinematics (“EEOIK”)

In some embodiments, an inverse kinematics method that utilizes an overridden end effector is used to solve a position and orientation of a simulated full limb. An end effector may be overridden by arbitrarily and artificially altering its position and orientation. This may be useful when rendering a full body avatar for a user having an amputated limb or body part. For instance, tracking data corresponding to an end effector of the amputated limb may be overridden by lengthening or extending the end effector to a new position and orientation. The artificially and arbitrarily extended end effector allows the VR engine to render animations for a complete limb from an amputated limb's tracking data.

A position and orientation of an end effector may be overridden using a linkage, a tether, a bounding box, or some other type of accessed constraint. A linkage, tether, or bounding box may fix two limbs or body parts according to a distance, an angle, or some combination thereof or may constrain two limbs or body parts within the boundaries of a bounding box, whereby the position and orientation of a tracked limb's end effector may determine what position and orientation a virtual simulated full limb's end effector is overridden to. For instance, a linkage or a tether may establish a minimum distance, a maximum distance, or some combination thereof between two limbs. As a tracked limb is tracked as moving relative to a virtual simulated full limb, the minimum and/or maximum distance thresholds may trigger a virtual simulated full limb to follow or be repelled by the tracked limb, whereby the tracked limb's end effector determines the overridden position and orientation of a simulated full limb's end effector. In another example, a linkage or tether establishes one or more catch angles between a tracked limb and a simulated full limb, whereby rotations of the tracked limb are translated into motion of a simulated full limb at the catch angles. In these examples, tracking data indicating movement of the tracked limb may not be translated to the animations of a virtual simulated full limb until the linkage or tether has reached its maximum distance or angle between the two limbs, after which point a simulated full limb may trail behind or be repelled by the movements of the tracked limb. In one example, a user is provided with a set of num-chucks in virtual reality, whereby the chain between the grips establishes a maximum distance between the hand of the tracked limb and the hand of a virtual simulated full limb, an interaction between the chain and the hand grips establishes a maximum angle, and the size of the hand grips establishes a minimum distance. In this example, the movements of the tracked limb are translated to movements of a virtual simulated full limb when any one of these thresholds is met, thereby enabling the position and orientation of the tracked limb's end effector to at least partially determine the overridden position and orientation of a virtual simulated full limb's end effector. A bounding box may establish a field of positions that a virtual simulated full limb can occupy relative to a tracked limb. (the “bounding box” description could benefit from further elucidation).

A position and orientation of an end effector may be overridden using a physical prop, a virtual prop, or both. A prop may fix the relative position of two end effectors. For instance, a prop may have two grip or contact points, whereby tracking data indicating movements of one grip point or one contact point determines a position and orientation of the second grip or contact point. A prop such as this may beneficially provide the illusion that an amputee is in control of their virtual simulated full limb. For instance, an amputee contacting a first grip or contact point of the prop will be provided with a visual indication of where their amputated limb should be positioned and how it should be orientated, e.g., as gripping or contacting the second grip or contact point. As an amputee instructs their intact limb to move, the prop will move and alter the position and orientation of a virtual simulated full limb. Once an amputee understands how the prop moves the second grip or contact point, they will be able to predict the movement animations the VR engine provides for a virtual simulated full limb based on the movements they make with their intact limb. Once an amputee can predict the corresponding movements, they can instruct their amputated limb to make those same movements and the VR engine will beneficially provide animations of a virtual simulated full limb making those same movements. As such, the prop provides predictable animations for a virtual simulated full limb that allow an amputee to feel a sense of control over their simulated full limb.

A prop may provide animations for a virtual simulated full limb using a modified inverse kinematics method. The modified inverse kinematics method may utilize a tracked limb with complete tracking data including an end effector, a virtual simulated full limb with incomplete tracking data (e.g., tracking data available only from a remaining portion of a limb, if at all), and a prop having two grip or contact points. The method may assign the tracked end effector as gripping or contacting a first section of the prop. Movements of the tracked end effector may be translated into movements of the prop.

A second section of the prop may serve as an overridden end effector for the tracked limb's amputated partner. For example, tracking data for an amputated limb's end effector that is communicated to the VR engine may be arbitrarily and artificially overridden such that the end effector is reassigned to the second section of the prop. The position and orientation of a virtual simulated full limb may then be solved using the second section of the prop as an end effector, while the position and orientation of the tracked limb may be solved using the end effector indicated by the tracking data. This allows an intact limb to effectively control the position of an animated virtual simulated full limb by manipulating the position of the prop and thereby provides a sense of volition over the animated virtual simulated full limb that can help alleviate phantom limb pain. A modified inverse kinematics method such as this may be referred to as an end effector override inverse kinematics (“EEOIK”) method. In one example, the VR engine receives tracking data indicating that a tracked limb is contacting a first contact point of an object and the VR engine then extends the end effector of the simulated full limb using the EEIOK method such that is artificially extends to a second contact point on the object. The tracking data may then directly drive animations for both the tracked arm and the prop, and the tracking data may indirectly drive the animations of a virtual simulated full limb through the prop.

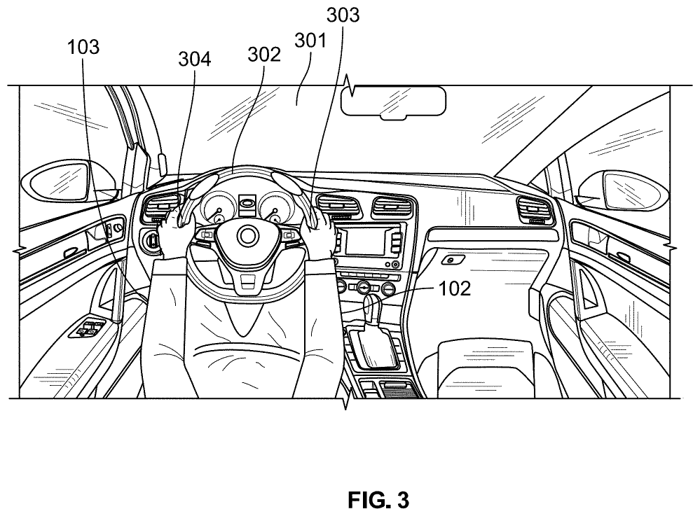

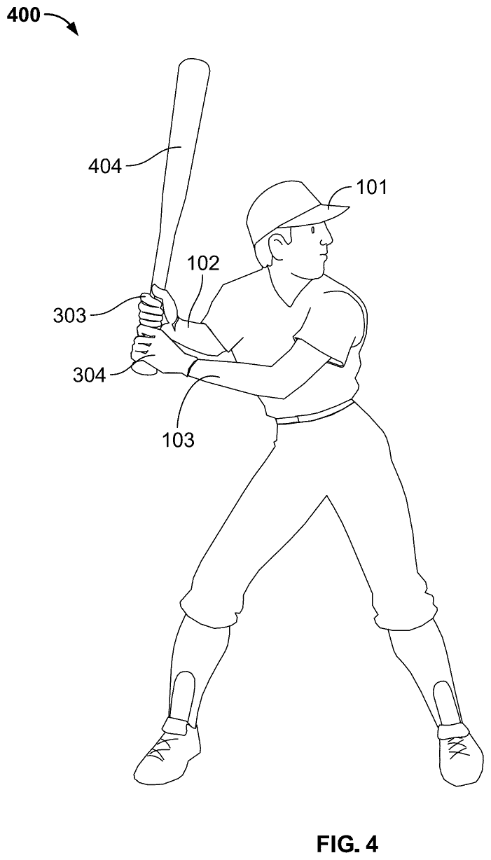

FIG. 3 is an illustrative depiction of a virtual reality driving activity, in accordance with some embodiments of the disclosure. For example, FIG. 3 illustrates an example of a virtual reality driving activity 301 that utilizes a steering wheel 302 as a prop. The steering wheel 302 may have a first section 303 and a second section 304 which are predefined gripping positions for each hand. When a user grips either of the predefined gripping sections with their tracked hand, a virtual simulated full limb will be animated as gripping the other section. As illustrated in FIG. 3, a user has gripped the steering wheel 302 at the first section 303 with their tracked arm 102 and a virtual simulated full arm 103 has been animated as gripping the second section 304. The steering wheel 302 fixes the distance and relative orientation between the tracked limb and a virtual simulated full limb. As the tracked limb 102 moves the steering wheel 302, it determines the position and orientation of a virtual simulated full arm 103. This allows the tracking data for the tracked limb to drive the animations of the tracked limb, the prop, and a virtual simulated full limb. For instance, a position and orientation of the tracked arm 102 may be solved using inverse kinematics that assigns the hand as an end effector and a position and orientation of a virtual simulated full arm 103 may be solved using EEOIK that assigns the second grip position of the steering wheel 302 as an overridden end effector.

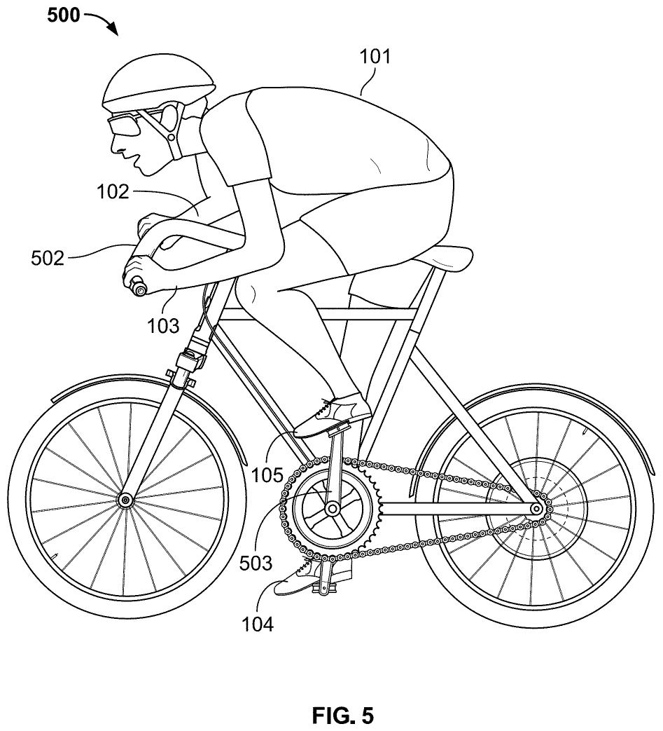

FIG. 4 is an illustrative depiction of a virtual reality baseball activity, in accordance with some embodiments of the disclosure. For example, FIG. 4 illustrates an example of a virtual reality baseball activity 400 that utilizes a baseball bat 404 as a prop. Baseball bat 404 may have a first section 303 and a second section 304 that are predefined gripping positions for each hand. When the VR engine receives tracking data indicating that a user has gripped either of the predefined gripping sections with their tracked hand, the VR engine may animate a virtual simulated full limb as gripping the other section. As illustrated in FIG. 4, an avatar 101 has been rendered with a baseball bat 404 gripped by a tracked arm 102 at a first section 303 and gripped by a virtual simulated full arm 103 at a second section 304. Baseball bat 404 fixes the distance and relative orientation between the tracked arm 102 and a virtual simulated full arm 103. As a user swings the baseball bat 404 with their intact arm, they can easily predict and anticipate a corresponding motion for their virtual simulated full limb. This predictability allows a user to instruct their simulated full limb to make the expected and predicted movements and the VR engine will supplement these volitions with animations of a virtual simulated full limb making those same expected and predicted movements, whereby the VR engine will elicit in a user a sense of control over a simulated full limb.

FIG. 5 illustrates an example of a virtual reality biking activity 500 that utilizes handlebars 502 as a prop, pedals 503 as a prop, or both. In such a case, the prop provides a first contact point and a second contact point. When tracking data for a tracked limb indicates that its end effector has contacted either the first contact point or the second contact point the VR engine animates a virtual simulated full limb as contacting the other contact point. As the tracked limb moves, it moves the prop, which in turn moves a virtual simulated full limb.

In an example illustrated by FIG. 5, a user has gripped the handlebars 502 at a first section with their tracked arm 102 and the VR engine has provided an animation of a virtual simulated full arm 103 gripping the handle bars 502 at a second section. The position and orientation of the tracked arm 102 may be solved with inverse kinematics using the tracking data for the hand of the tracked arm 102 as an end effector and the position and orientation of a virtual simulated full arm 103 may be solved with EEOIK using the second contact point of the prop as an overridden end effector. In this way, the position and orientation of the tracked arm 102 drives the position and orientation of the prop and a virtual simulated full arm 103. As a user moves their tracked arm 102, the handlebars 502 move, which in turn moves a virtual simulated full arm 103 in a predictable and controllable manner. In this example, a pivot point of the handlebars 502 is at a center point of the handlebars 502. The pivot point of a prop may be restricted to only allow forward and backward movements across a pivot point, while movements along different axes may be directly translated across, in this case the handlebars 502, without any pivoting.

Also in an example illustrated by FIG. 5, an avatar 101 has been rendered with a prop in the form of bike pedals 503 contacted by a tracked leg 104 on one pedal and contacted by a virtual simulated full leg 105 on another pedal. In this example, the VR engine may receive tracking data that a tracked foot is contacting a first pedal of a bicycle, whereby the tracked foot serves as an end effector for that leg. The VR engine may then artificially and arbitrarily extend the end effector of a simulated full leg, across the two crank arms and the spindle connecting the two pedals, such that the simulated full leg is positioned as contacting a second pedal of the bicycle. During motion, the tracked limb and a virtual simulated full limb may traverse the path of a conic section that rotates about a common axis. Like other props, the pedal 503 allows a user to accurately predict what movements his or her virtual simulated full limb will make and instruct it accordingly.

### Synchronous Activities

The modified inverse kinematics method of the present disclosure may be customized for specific types of activity. Activities may require symmetrical movement, relational movement, aligned movement, tethered movement, patterned movements, item gripping, item manipulation, or specific limb placement. Each type of activity may utilize a different inverse kinematics method to animate a virtual simulated full limb that moves in a predictable and seemingly controlled manner to perform a given activity for rehabilitation. The efficacy of a particular method may vary from activity to activity. In some instances, multiple methods may be weighted and balanced to determine virtual simulated full limb animations.

Humans are adept at moving a single limb carefully and deliberately while its partner limb remains stationary. However, it is often difficult to move two partner limbs, e.g., two arms, two hands, two feet, two legs, etc., without some form of synchronization. This is one reason why it is often comically difficult to rub one's belly and pat one's head simultaneously. A specific type of synchronization each limb moves with may depend on the activity being performed. When someone kicks a soccer ball, one foot plants itself for balance and the other kicks the ball, when someone shoots a basketball two hands work in sync, when someone rides a bike, flies a kite, paddles a kayak, claps, sutures, knits, or even dances their limbs move in synchronization. Often, the movements of one partner limb can determine the corresponding movement required by the other partner limb, and at the very least, partner limbs can inform what movements the other limb ought to make.

The modified inverse kinematics solution disclosed herein may utilize information about the activity being performed, e.g., what kind of symmetry frequently occurs or is required to occur, to assist in positioning a virtual simulated full limb. In some instance, the type of symmetry may fix animations such that the tracked limb determines the movement of a virtual simulated full limb. Alternatively, the type of symmetry may only influence or inform the animations that are provided for a virtual simulated full limb. In some embodiments, each activity may feature a predefined movement pattern, whereby the animations provided for a user may be modulated by the predefined movement pattern. For example, tracking data that traverses near the predefined movement pattern may be partially adjusted to more closely align with the trajectory of the predefined movement pattern or the tracking data may be completely overridden to the trajectory of the predefined movement pattern. This may be useful for increasing the confidence of a user and may also help nudge them towards consistently making the desired synchronous movements.

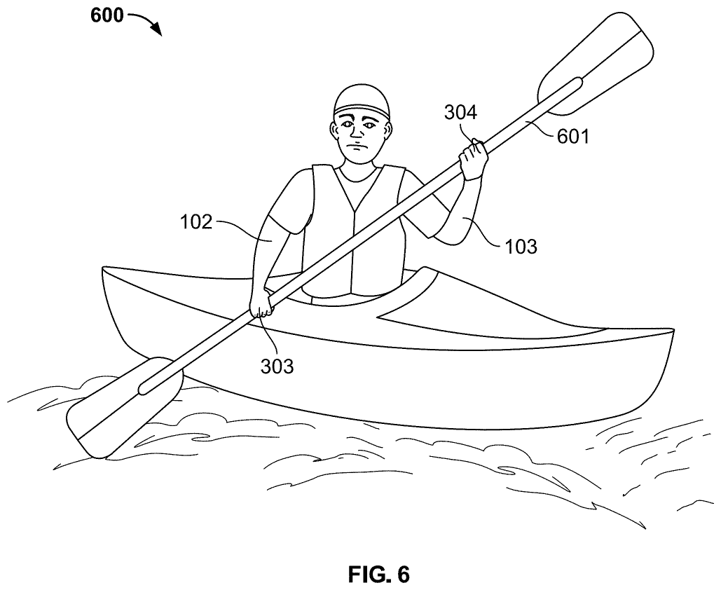

FIG. 6 is an illustrative depiction of a virtual reality kayaking activity, in accordance with some embodiments of the disclosure. For instance, FIG. 6 illustrates an example of a virtual reality kayaking activity 600 that requires synchronous movements of a kayak paddle 601 to propel the kayak. In this activity, the VR engine may receive tracking data indicating that an end effector of a tracked arm 102, e.g., a hand, has gripped a first section 102 and the VR engine may then animate a hand of a virtual simulated full arm 103 as gripping the second section 103. As a user manipulates the kayak paddle 601 with their tracked arm 102, their virtual simulated full arm 103 may be animated as making corresponding, synchronous movements. Animations may be generated using a combination of a traditional inverse kinematics method that utilizes tracking data of a hand as an end effector of the tracked arm 102 and a EEOIK method that utilizes a section of the kayak paddle 601 as an arbitrarily and artificially extended end effector of a virtual simulated full arm 103. In some instances, the VR engine may override tracking data completely or partially to animate the kayak as making a smooth motion according to a predefined movement pattern despite tracking data indicating a less precise movement. This may help a user learn the proper movements and at times make a user believe they are performing the proper synchronous movements even if they are not. In some embodiments, the kayak paddle 601 may have a pivot point at its center point. The pivot point may be fixed or may be able to traverse limited translation. The pivot point may simplify the dexterity required by a user to control the kayak paddle 601 with only one hand.

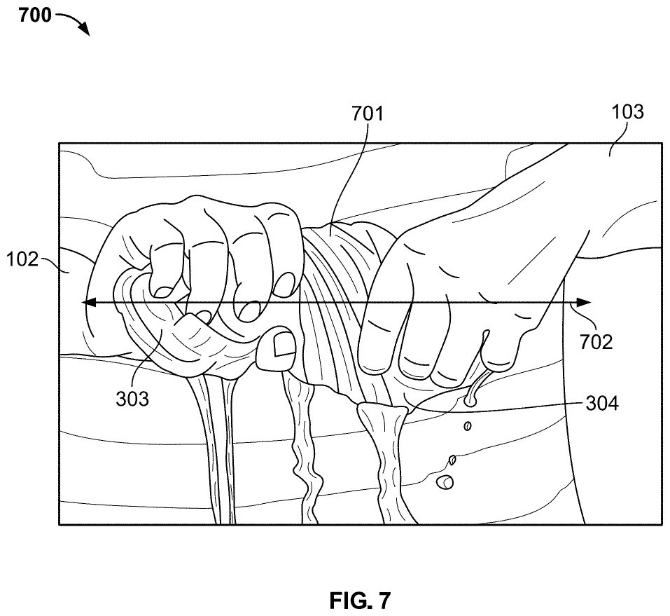

FIG. 7 is an illustrative depiction of a virtual reality towel wringing activity, in accordance with some embodiments of the disclosure. For example, FIG. 7 illustrates an example of a virtual reality towel wringing activity 700 that requires synchronous twists of a wet towel 701. In this activity, a user grips a wet towel 701 and rotates their wrists along a common axis 702. Unlike pronation and supination that rotates the wrist along an axis parallel with the forearm, the rotation of the wrist when wringing the wet towel 701 is along an axis that is perpendicular to the forearm. In this example, the axis of rotation is established by a length of the wet towel 701 as indicated by the common axis 702. The wet towel 701 may feature a first and second grip point. A tracked arm 102 gripping either the first or second grip point may result in an animation of a virtual simulated full arm 103 gripping the other of the two portions. In the example illustrated by FIG. 7, the VR engine has rendered an avatar 101 with a tracked arm 102 gripping a first section 303 of the wet towel 701 and a virtual simulated full arm 103 gripping a second section 304 of the wet towel 701. Tracking data indicating that the tracked arm 102 is rotating along the common axis 702 in one direction may result in an animation of a simulated full arm 103 rotating along the common axis 702 in the opposite direction. This will generate torsion in the wet towel 701 that releases water. In one example, the hands do not rotate along an identical axis, but rather rotate along two separate axes that are each offset by, e.g., 1 to 45 degrees relative to common axis 702 such that the axes intersect above and between both hands. Like other example described herein, the tracked arm 102 may be solved using tracking data as an end effector, while a portion of the prop, in this case a section of the wet towel 701, serves as an end effector, whereby the position and orientation of both arms are solvable using their respective end effectors in a EEOIK method.

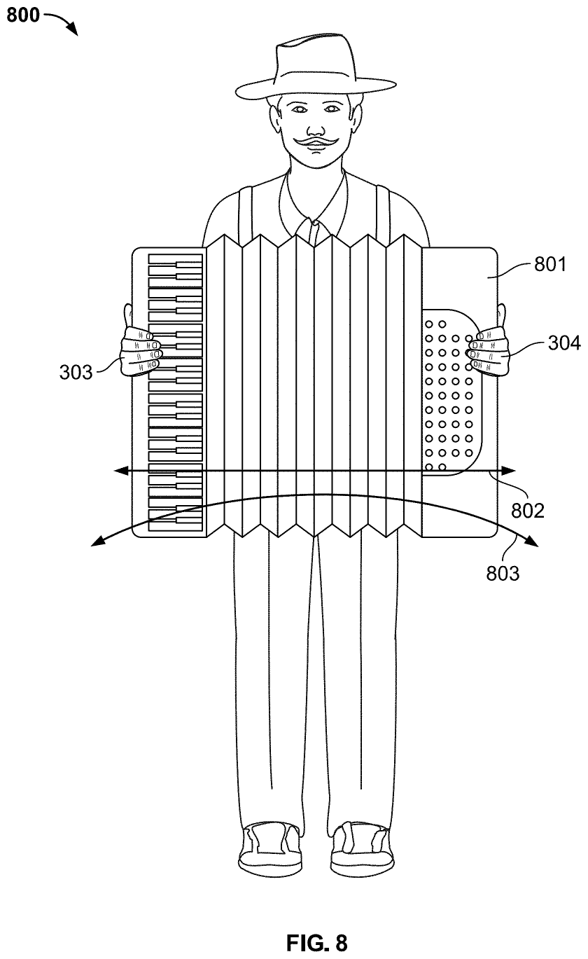

FIG. 8 is an illustrative depiction of a virtual reality accordion playing activity, in accordance with some embodiments of the disclosure. For instance, FIG. 8 illustrates an example of a virtual reality accordion playing activity 800 that requires the synchronous manipulation of an accordion 801. In this activity, a user grips an accordion 801 with their tracked limb on either a right-hand side 303 or a left-hand side 304, while a virtual simulated full limb is animated as gripping the other of the two sides. The grip of the accordion orientates the thumbs of a user towards the sky.

When the VR engine receives tracking data indicating that the tracked arm is moving away from the body's midline or towards the body's midline, a simulated full arm is animated as moving in the same direction such that the accordion is stretched and compressed. This type of movement may traverse a linear axis 802. This type of rule base symmetry is similar to the type of animations that would be animated with a virtual mirror at a user's midline, whereby an arm moving towards the mirror generates mirrored data of a virtual simulated full limb moving towards the mirror and vice versa. In addition to this linear axis 802, a user may move the accordion along a curved axis 803. For instance, if the VR engine receives tracking data indicating that the tracked limb is moving down and the thumb is rotating from an up position to an out position, then a mirrored copy of this movement may be animated for a simulated full limb, such that the accordion traverse a curved axis 803 such as illustrated in FIG. 8. In this example, a user may move their tracked limb along a curved axis and be provided with movement animations for their virtual simulated full limb that are easy to predict.

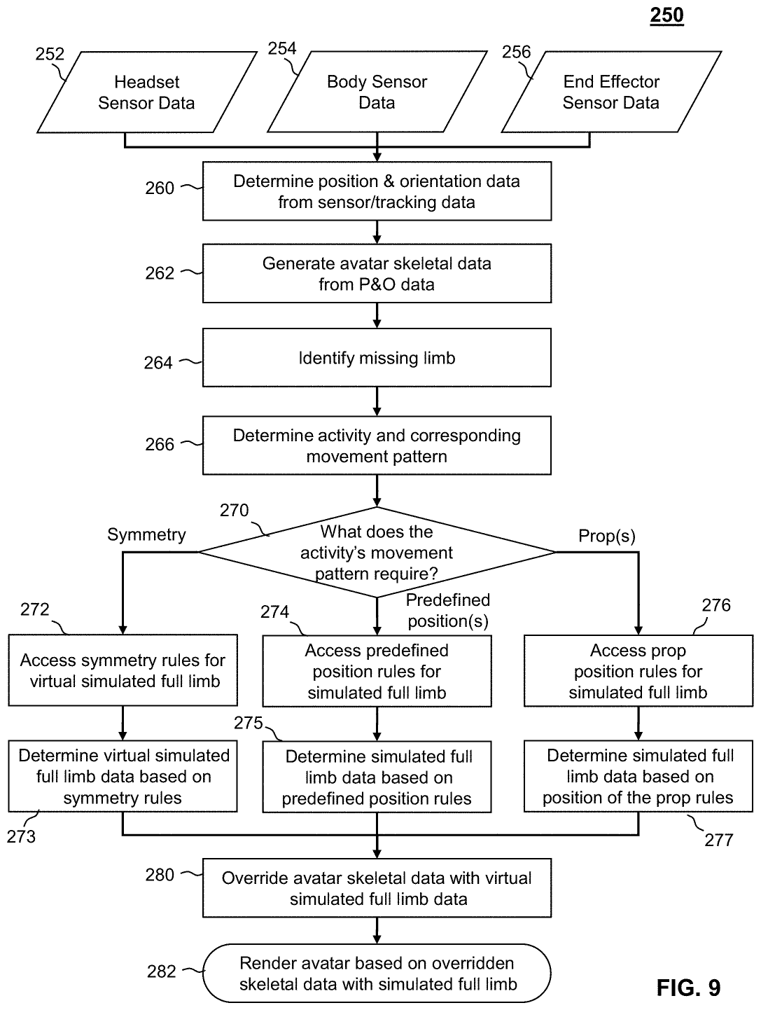

FIG. 9 depicts an illustrative flow chart of a process for overriding position and orientation data with a simulated full limb, in accordance with some embodiments of the disclosure. Generally, process 250 of FIG. 9 includes steps for identifying a missing limb(s), determining movement patterns for a particular (VR) activity, applying rules corresponding to the determined movement pattern to determine simulated full limb position and orientation data, and overriding avatar skeletal data to generate and render avatar skeletal data with a simulated full limb.

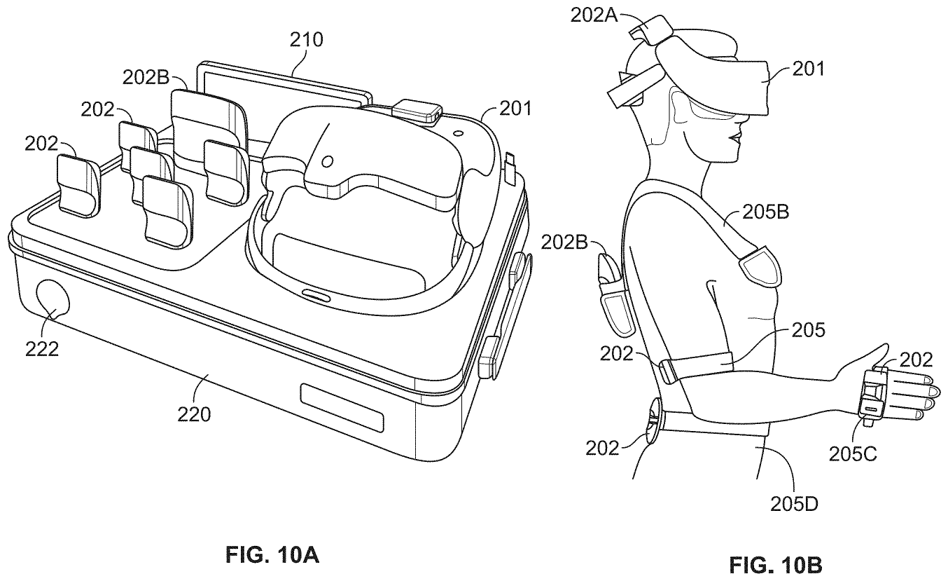

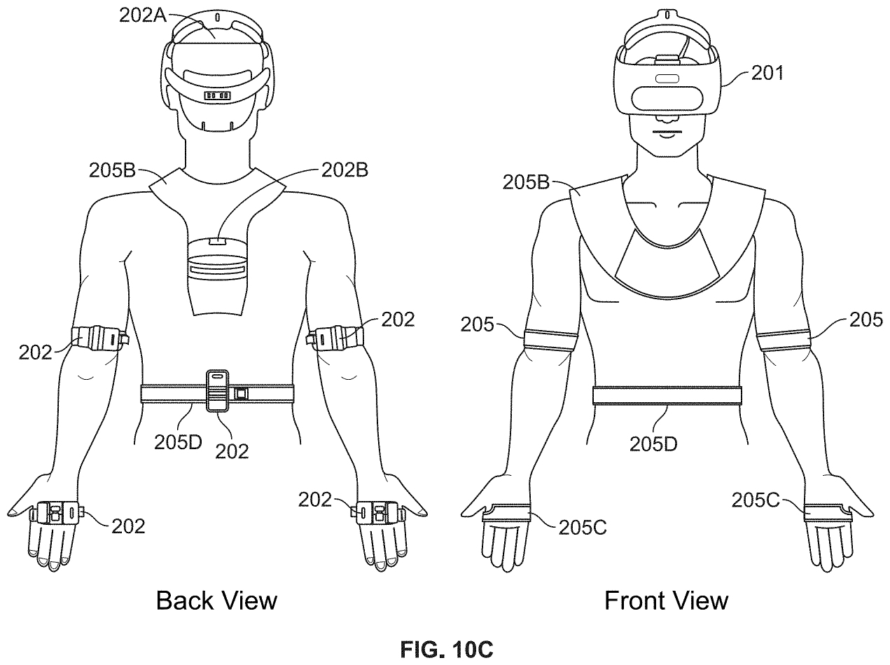

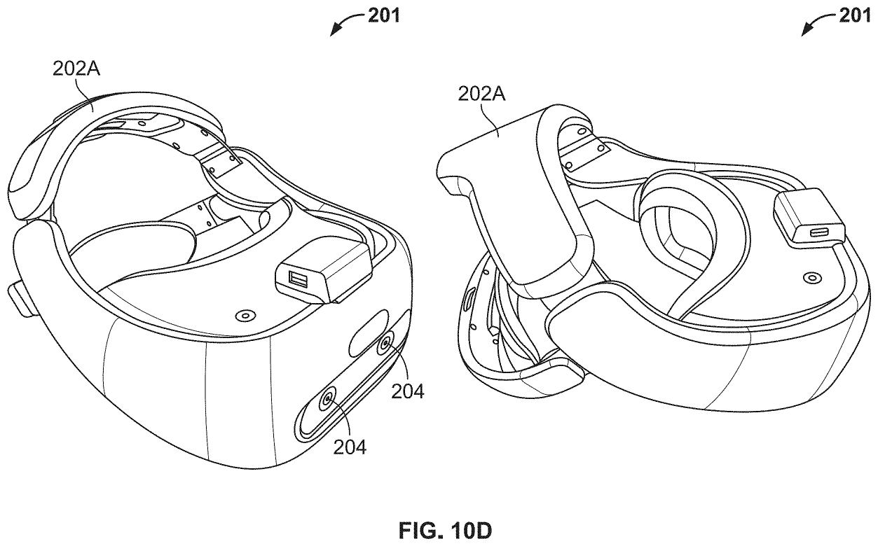

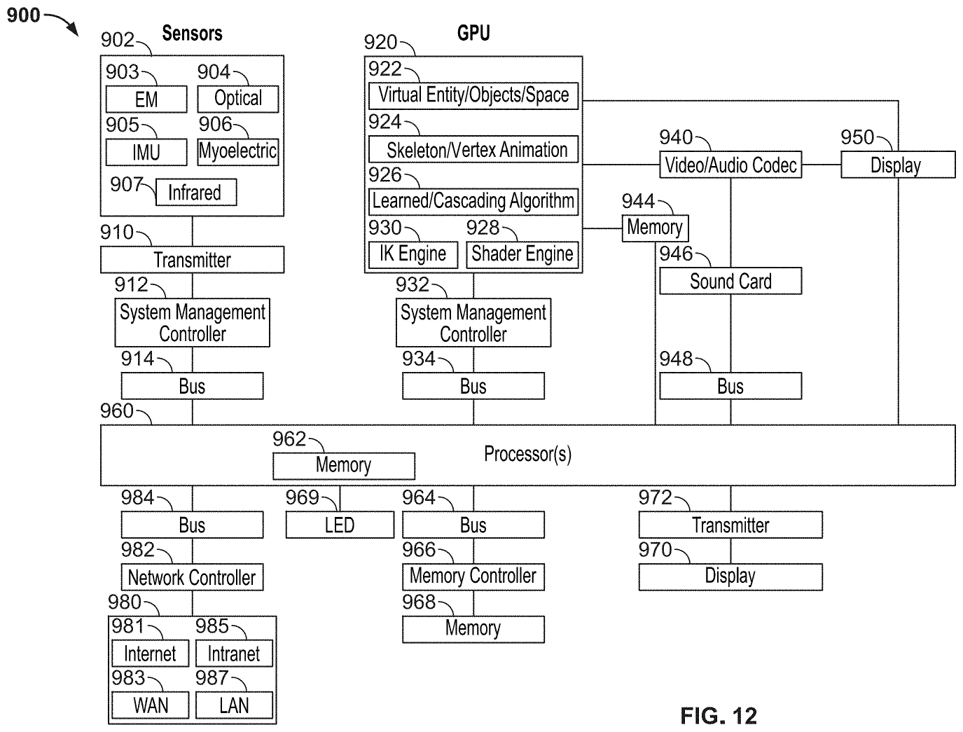

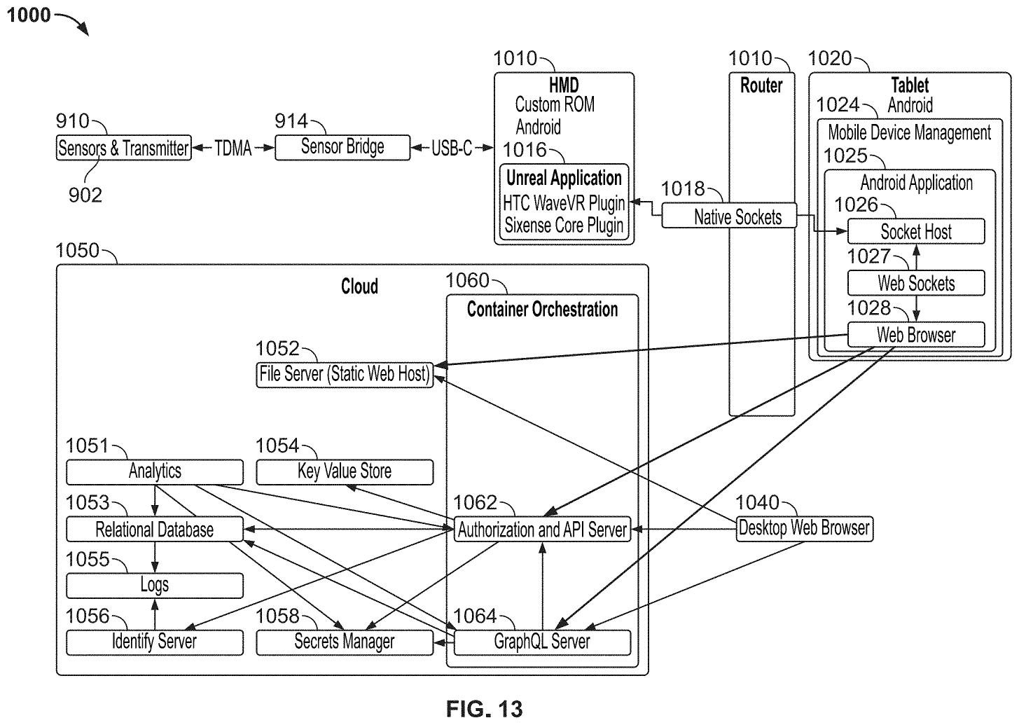

Some embodiments may utilize a VR engine to perform one or more parts of process 250, e.g., as part of a VR application, stored and executed by one or more of the processors and memory of a headset, server, tablet and/or other device. For instance, VR engine may be incorporated in one or more of head-mounted display 201 and clinician tablet 210 of FIGS. 10A-D and/or the systems of FIGS. 12-13. A VR engine may run on a component of a tablet, HMD, server, display, television, set-top box, computer, smart phone, or other device.

At input 252, headset sensor data may be captured and input into, e.g., a VR engine. Headset sensor data may be captured, for instance, by a sensor on the HMD, such as sensor 202A on HMD 201 as depicted in FIGS. 10A-D. Sensors may transmit data wirelessly or via wire, for instance, to a data aggregator or directly to the HMD for input. Additional sensors, placed at various points on the body, may also measure and transmit/input sensor data.

At input 254, body sensor data—e.g., hand, arm, back, legs, ankles, feet, pelvis and other sensor data—may be captured and input in the VR engine. Hand and arm sensor data may be captured, for instance, by sensors affixed to a patient's hands and arms, such as sensors 202 as depicted in FIGS. 10A-C and 11A-C. Sensor data from each sensor on each corresponding body part may be transmitted and input separately or together.

At input 256, data from sensors placed on prosthetics and end effectors may be captured and input into the VR engine. Generally, sensors placed on prosthetics and end effectors may be the same as sensors affixed to a patient's body parts, such as sensors 202 and 202B as depicted in FIGS. 10A-C and 11A-C. In some cases, sensors placed on a prosthetic arm or an end effector for a hand may be at positioned at the same distance as a body part or close by. For instance, sensors placed on prosthetics and end effectors may not always be placed in a typical position and may be positioned as close to a normal sensor position—e.g., positioned on the prosthetic body part or end effector as if placed on a unamputated body part. Sensor data from each of the sensors placed on amputated limbs may be transmitted and input like any other body sensor.

With each of inputs 252, 254, and 256, data from sensors may be input into the VR engine. Sensor data may comprise location and rotation data in relation to a central sensor such as a sensor on the HMD or a sensor on the back in between the shoulder blades. For instance, each sensor may measure a three-dimensional location and measure rotations around three axes. Each sensor may transmit data at a predetermined frequency, such as 60 Hz or 200 Hz.

At step 260, the VR engine determines position and orientation (P&O) data from sensor data. For instance, data may include a location in the form of three-dimensional coordinates and rotational measures around each of the three axes. The VR engine may produce virtual world coordinates from these sensor data to eventually generate skeletal data for an avatar. In some embodiments, sensors may feed the VR engine raw sensor data. In some embodiments, sensors may input filtered sensor data into sensor engine 620. For instance, the sensors may process sensor data to reduce transmission size. In some embodiments, sensor 202 may pre-filter or clean “jitter” from raw sensor data prior to transmission. In some embodiments, sensor 202 may capture data at a high frequency (e.g., 200 Hz) and transmit a subset of that data, e.g., transmitting captured data at a lower frequency. In some embodiments, VR engine may filter sensor data initially and/or further.

At step 262, the VR engine generates avatar skeletal data from the determined P&O data. Generally, a solver employs inverse kinematics (IK) and a series of local offsets to constrain the skeleton of the avatar to the position and orientation of the sensors. The skeleton then deforms a polygonal mesh to approximate the movement of the sensors. An avatar includes virtual bones and comprises an internal anatomical structure that facilitates the formation of limbs and other body parts. Skeletal hierarchies of these virtual bones may form a directed acyclic graph (DAG) structure. Bones may have multiple children, but only a single parent, forming a tree structure. Two bones may move relative to one another by sharing a common parent.

At step 264, the VR engine identifies the missing limb, e.g., the amputated limb that will be rendered as a virtual simulated full limb. In some embodiments, identifying the missing limb may be performed prior to generating avatar skeletal data or even receiving data. For instance, a therapist (or patient) may identify a missing limb in a profile or settings prior to therapy or VR games and activities, e.g., when using an “amputee mode” of the VR application. In some embodiments, identifying the missing limb may be performed by analyzing skeletal data to identify missing sensors or unconventionally positioned sensors. In some embodiments, identifying the missing limb may be performed by analyzing skeletal movement data to identify unconventional movements.

At step 266, the VR engine determines which activity (e.g., game, task, etc.) is being performed and determines a corresponding movement pattern. An activity may require, e.g., synchronized movements, symmetrical movement, relational movement, aligned movement, tethered movement, patterned movements, item gripping, item manipulation, and/or or specific limb placement. For instance, if the activity is a virtual mirror, like activity depicted in FIG. 1, it will comprise symmetrical movement. Activities depicted in FIG. 2B may comprise parallel movements. Activities depicted in 2C-D may comprise symmetrical movements and/or parallel movements. Activities depicted in FIGS. 3-8 may comprise relational movement, tethered movement, item gripping and/or item manipulation. In some embodiments, application data (e.g., games and activities) may be stored at the headset, e.g., HMD 101 of system 1000 depicted in FIG. 13. In some embodiments, application data may be stored at on a network-connected server, e.g., cloud 1050 and/or file server 1052 depicted in FIG. 13. Movement patterns associated with a game, activity, and/or task may be stored with the application or separately and linked.

At step 270, the VR engine determines what rules the activity's movement pattern requires. Some synchronized movements and/or symmetrical movements may require symmetry rules. For example, generating simulated full limb movements with a virtual mirror, e.g., depicted in FIG. 1, may require symmetry rules. Generating simulated full limb movements regarding squeezing an accordion, e.g., depicted in FIG. 8, may require symmetry rules. Some synchronized movements, relational movements, tethered movements, and/or gripping movements may require predefined position rules. For example, generating simulated full limb movements with a steering wheel activity, e.g., depicted in FIG. 3, and/or biking, e.g., depicted in FIG. 5, may require predefined position rules. Some synchronized movements, relational movements, tethered movements, item manipulation movements, and/or gripping movements may require prop position rules. For instance, generating simulated full limb movements with swinging a baseball bat, e.g., depicted in FIG. 4, or kayaking, e.g., depicted in FIG. 6, may require prop position rules. Some movements may require one or more of symmetry rules, predefined position rules, and/or prop position rules.

If the VR engine determines that the activity's movement pattern requires symmetry at step 270, the VR engine accesses symmetry rules for a simulated full limb at step 272. Symmetry rules may describe rules to generate position and orientation data for a simulated full limb in terms of symmetrical movement of an opposite (full) limb. For example, the VR engine may determine that symmetry rules may be required to generate simulated full limb movements for activity like a virtual mirror, e.g., depicted in FIG. 1. Generating simulated full limb movements regarding squeezing an accordion, e.g., depicted in FIG. 8, may require symmetry rules. Symmetry rules may be required for rendering some synchronized movements and/or symmetrical movements. In some embodiments, symmetry rules may comprise rules for parallel movement, opposite movement, relational movement, and/or other synchronized movement. In some embodiments, rules (e.g., symmetry rules) may be accessed as part of local application data. In some embodiments, rules may be accessed as part of remote (cloud) application data. In some embodiments, rules may be accessed separately from application data, e.g., as part of input instructions and/or accessibility instructions for processing.

At step 273, the VR engine determines simulated full limb data based on symmetry rules. For example, the VR engine may generate simulated full limb movements for activity like a virtual mirror, e.g., depicted in FIG. 1, by reflecting P&O data of a full limb over an axes (or plane) to generate P&O data for a simulated full limb. In some embodiments, the VR engine may generate simulated full limb movements regarding squeezing an accordion, e.g., depicted in FIG. 8, by reflecting P&O data of a full limb over an axes, along a curved axis (following an accordion squeeze shape) to generate P&O data for a simulated full limb.

If the VR engine determines that the activity's movement pattern requires a predefined position at step 270, the VR engine accesses predefined position rules for a simulated full limb at step 274. For example, the VR engine may determine that predefined position rules may be required to generate simulated full limb movements for, e.g., a steering wheel activity (depicted in FIG. 3) or a biking activity (depicted in FIG. 5). Predefined position rules may be required for some synchronized movements, relational movements, tethered movements, and/or gripping movements. The VR engine can adjust positions and orientations based on other body parts, as necessary.

At step 275, the VR engine determines simulated full limb data based on predefined position rules. For example, the VR engine may generate simulated full limb movements for activity like a turning a steering wheel, e.g., depicted in FIG. 3, by translating P&O data of a full limb to generate P&O data for a simulated full limb on a particular position of the steering wheel. The VR engine may generate a right hand gripping the wheel at 2 o'clock when the left hand grips the wheel at 10 o'clock and adjust the positions and orientations as necessary when a limb is detected to move. In some embodiments, the VR engine may generate simulated full limb movements for activity like a pedaling a bicycle, e.g., depicted in FIG. 5, by translating P&O data of a full leg limb to generate P&O data for a virtual simulated full leg limb on a particular position of the corresponding pedal. If the full leg limb moves the virtual bicycle pedal from top to bottom, the other pedal and the virtual simulated full leg limb should follow, e.g., according to predefined position rules, and adjust the positions and orientations as necessary. Likewise, when animating an avatar wringing a washcloth, as a full hand clenches and turns one way, position rules would instruct the virtual simulated full hand to squeeze and rotate the opposite direction. The VR engine can adjust positions and orientations based on other body parts, as well.

If the VR engine determines that the activity's movement pattern requires a prop at step 270, the VR engine accesses prop position rules for a simulated full limb at step 276. For instance, prop position rules may be required to generate simulated full limb movements for activities like swinging a baseball bat (depicted in FIG. 4) and/or kayaking (depicted in FIG. 6). Prop position rules may be required for activities with a (virtual) prop or prop-like movement, e.g., some synchronized movements, relational movements, tethered movements, item manipulation movements, and/or gripping movements.

At step 277, the VR engine determines simulated full limb data based on position of the prop rules. For example, the VR engine may generate simulated full limb movements for activity like a turning a swinging a baseball bat, e.g., depicted in FIG. 4, by translating P&O data of a full limb to generate P&O data for a simulated full limb based on the customary position of the hand gripping the bat. For a right-handed batter, the VR engine may generate a virtual left hand gripping the bat at the base of the bat handle and when the right hand grips the virtual baseball bat a bit higher on the handle. The VR engine can adjust position and orientation data of the virtual left hand as the right hand swings the bat through. In some embodiments, the VR engine may generate simulated full limb movements for activity like kayaking, e.g., depicted in FIG. 6, by translating P&O data of a full arm limb to generate P&O data for a virtual simulated full arm limb on a particular opposite position of the kayak. If the full arm limb paddles the virtual water from forward to backward, the other kayak paddle should correspondingly move in the air backward to forward. The VR engine can adjust positions and orientations based on other body parts, as necessary.

At step 280, after performance of step 273, 275, and/or 277, the VR engine overrides avatar skeletal data with simulated full limb data. In some embodiments, the VR engine may generate P&O data for a virtual simulated full limb based on the rule, which may be converted to skeletal data. For instance, simulated full limb position and orientation may be substituted for a body part with improper, abnormal, limited, or no sensor data or tracking data. For instance, using a symmetry rule, translated and adjusted left arm data may supplant right arm data. For example, using a predefined position rule, known position and orientation data for a left hand may supplant the received left hand P&O data. For instance, using a prop position rule, position and orientation data for an amputated left arm determined based on relation to P&O data for a full right arm, may supplant the received left arm P&O data. In some embodiments, the VR engine may generate skeletal data based on the rule and not generate P&O data for a simulated full limb. In some embodiments, the VR engine may generate skeletal data for a simulated full limb based on kinematics and/or inverse kinematics.

At step 282, the VR engine renders an avatar, with a simulated full limb, based on overridden skeletal data. For example, the VR engine may render and animate an avatar using both arms to kayak, or both legs to bicycle, or both hands to steer a car.

FIGS. 10A-D are diagrams of an illustrative system, in accordance with some embodiments of the disclosure. A VR system may include a clinician tablet 210, head-mounted display 201 (e.g., HMD or headset), small sensors 202, and large sensor 202B. Large sensor 202B may comprise transmitters, in some embodiments, and be referred to as wireless transmitter module 202B. Some embodiments may include sensor chargers, router, router battery, headset controller, power cords, USB cables, and other VR system equipment.

Clinician tablet 210 may be configured to use a touch screen, a power/lock button that turns the component on or off, and a charger/accessory port, e.g., USB-C. For instance, pressing the power button on clinician tablet 210 may power on the tablet or restart the tablet. Once clinician tablet 210 is powered on, a therapist or supervisor may access a user interface and be able to log in; add or select a patient; initialize and sync sensors; select, start, modify, or end a therapy session; view data; and/or log out.

Headset 201 may comprise a power button that turns the component on or off, as well as a charger/accessory port, e.g., USB-C. Headset 201 may also provide visual feedback of virtual reality applications in concert with the clinician tablet and the small and large sensors.

Charging headset 201 may be performed by plugging a headset power cord into the storage dock or an outlet. To turn on headset 201 or restart headset 201, the power button may be pressed. A power button may be on top of the headset. Some embodiments may include a headset controller used to access system settings. For instance, a headset controller may be used only in certain troubleshooting and administrative tasks and not necessarily during patient therapy. Buttons on the controller may be used to control power, connect to headset 201, access settings, or control volume.

The large sensor 202B and small sensors 202 are equipped with mechanical and electrical components that measure position and orientation in physical space and then translate that information to construct a virtual environment. Sensors 202 are turned off and charged when placed in the charging station. Sensors 202 turn on and attempt to sync when removed from the charging station. The sensor charger acts as a dock to store and charge the sensors. In some embodiments, sensors may be placed in sensor bands on a patient. Sensor bands 205, as depicted in FIGS. 10B-C, are typically required for use and are provided separately for each patient for hygienic purposes. In some embodiments, sensors may be miniaturized and may be placed, mounted, fastened, or pasted directly onto a user.

As shown in illustrative FIG. 10A, various systems disclosed herein consist of a set of position and orientation sensors that are worn by a VR participant, e.g., a therapy patient. These sensors communicate with HMD 201, which immerses the patient in a VR experience. An HMD suitable for VR often comprises one or more displays to enable stereoscopic three-dimensional (3D) images. Such internal displays are typically high-resolution (e.g., 2880×1600 or better) and offer high refresh rate (e.g., 75 Hz). The displays are configured to present 3D images to the patient. VR headsets typically include speakers and microphones for deeper immersion.

HMD 201 is a piece central to immersing a patient in a virtual world in terms of presentation and movement. A headset may allow, for instance, a wide field of view (e.g., 110°) and tracking along six degrees of freedom. HMD 201 may include cameras, accelerometers, gyroscopes, and proximity sensors. VR headsets typically include a processor, usually in the form of a system on a chip (SoC), and memory. In some embodiments, headsets may also use, for example, additional cameras as safety features to help users avoid real-world obstacles. HMD 201 may comprise more than one connectivity option in order to communicate with the therapist's tablet. For instance, an HMD 201 may use an SoC that features WiFi and Bluetooth connectivity, in addition to an available USB connection (e.g., USB Type-C). The USB-C connection may also be used to charge the built-in rechargeable battery for the headset.

A supervisor, such as a health care provider or therapist, may use a tablet, e.g., tablet 210 depicted in FIG. 10A, to control the patient's experience. In some embodiments, tablet 210 runs an application and communicates with a router to cloud software configured to authenticate users and store information. Tablet 210 may communicate with HMD 201 in order to initiate HMD applications, collect relayed sensor data, and update records on the cloud servers. Tablet 210 may be stored in the portable container and plugged in to charge, e.g., via a USB plug.

In some embodiments, such as depicted in FIGS. 10B-C, sensors 202 are placed on the body in particular places to measure body movement and relay the measurements for translation and animation of a VR avatar. Sensors 202 may be strapped to a body via bands 205. In some embodiments, each patient may have her own set of bands 205 to minimize hygiene issues.

A wireless transmitter module (WTM) 202B may be worn on a sensor band 205B that is laid over the patient's shoulders. WTM 202B sits between the patient's shoulder blades on their back. Wireless sensor modules 202 (e.g., sensors or WSMs) are worn just above each elbow, strapped to the back of each hand, and on a pelvis band that positions a sensor adjacent to the patient's sacrum on their back. In some embodiments, each WSM communicates its position and orientation in real-time with an HMD Accessory located on the HMD. Each sensor 202 may learn its relative position and orientation to the WTM, e.g., via calibration.

The HMD accessory may include a sensor 202A that may allow it to learn its position relative to WTM 202B, which then allows the HMD to know where in physical space all the WSMs and WTM are located. In some embodiments, each sensor 202 communicates independently with the HMD accessory which then transmits its data to HMD 201, e.g., via a USB-C connection. In some embodiments, each sensor 202 communicates its position and orientation in real-time with WTM 202B, which is in wireless communication with HMD 201.

A VR environment rendering engine on HMD 201 (sometimes referred to herein as a “VR application”), such as the Unreal Engine™, uses the position and orientation data to create an avatar that mimics the patient's movement.

A patient or player may “become” their avatar when they log in to a virtual reality game. When the player moves their body, they see their avatar move accordingly. Sensors in the headset may allow the patient to move the avatar's head, e.g., even before body sensors are placed on the patient. A system that achieves consistent high-quality tracking facilitates the patient's movements to be accurately mapped onto an avatar.

Sensors 202 may be placed on the body, e.g., of a patient by a therapist, in particular locations to sense and/or translate body movements. The VR engine can use measurements of position and orientation of sensors placed in key places to determine movement of body parts in the real world and translate such movement to the virtual world. In some embodiments, a VR system may collect data for therapeutic analysis of a patient's movements and range of motion.

In some embodiments, systems and methods of the present disclosure may use electromagnetic tracking, optical tracking, infrared tracking, accelerometers, magnetometers, gyroscopes, myoelectric tracking, other tracking techniques, or a combination of one or more of such tracking methods. The tracking systems may be parts of a computing system as disclosed herein. The tracking tools may exist on one or more circuit boards within the VR system (see FIG. 12) where they may monitor one or more users to perform one or more functions such as capturing, analyzing, and/or tracking a subject's movement. In some cases, a VR system may utilize more than one tracking method to improve reliability, accuracy, and precision.

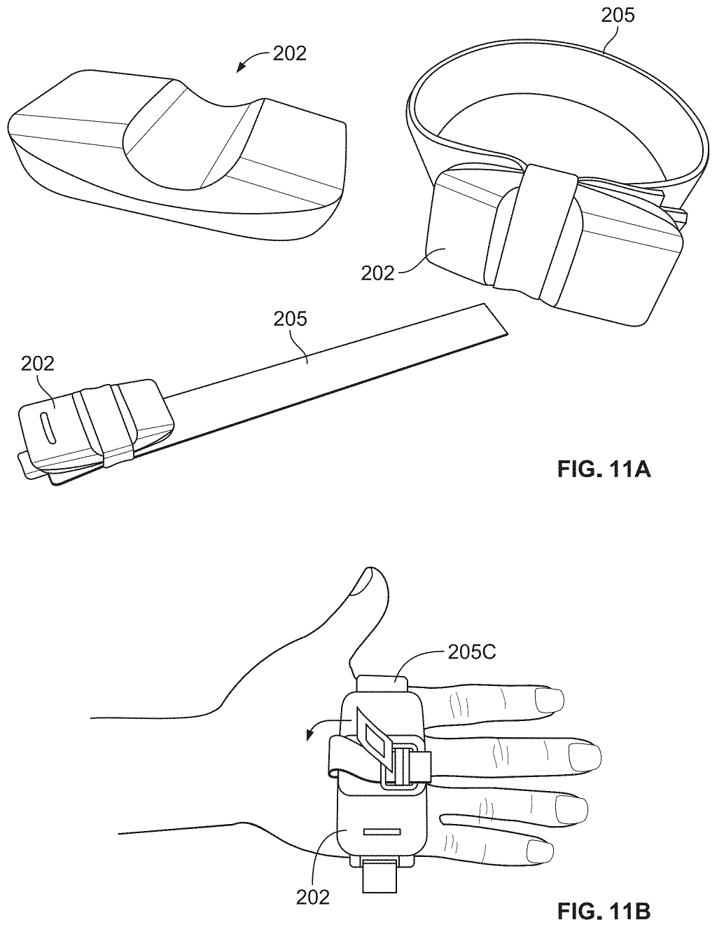

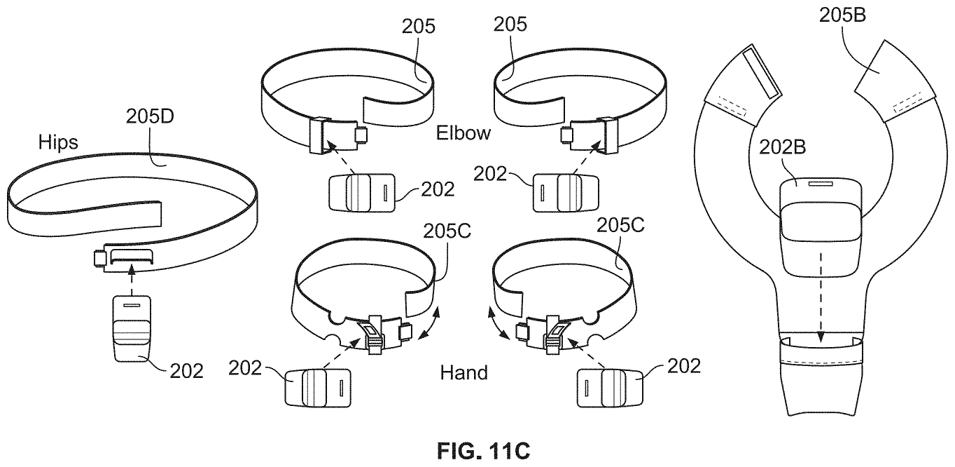

FIGS. 11A-C illustrate examples of wearable sensors 202 and bands 205. In some embodiments, bands 205 may include elastic loops to hold the sensors. In some embodiments, bands 205 may include additional loops, buckles and/or Velcro straps to hold the sensors. For instance, bands 205 for hands may require extra secureness as a patient's hands may be moved at a greater speed and could throw or project a sensor in the air if it is not securely fastened. FIG. 2C illustrates an exemplary embodiment with a slide buckle.

Sensors 202 may be attached to body parts via band 205. In some embodiments, a therapist attaches sensors 202 to proper areas of a patient's body. For example, a patient may not be physically able to attach band 205 to herself. In some embodiments, each patient may have her own set of bands 205 to minimize hygiene issues. In some embodiments, a therapist may bring a portable case to a patient's room or home for therapy. The sensors may include contact ports for charging each sensor's battery while storing and transporting in the container, such as the container depicted in FIG. 10A.

As illustrated in FIG. 11C, sensors 202 are placed in bands 205 prior to placement on a patient. In some embodiments, sensors 202 may be placed onto bands 205 by sliding them into the elasticized loops. The large sensor, WTM 202B, is placed into a pocket of shoulder band 205B. Sensors 202 may be placed above the elbows, on the back of the hands, and at the lower back (sacrum). In some embodiments, sensors may be used at the knees and/or ankles. Sensors 202 may be placed, e.g., by a therapist, on a patient while the patient is sitting on a bench (or chair) with his hands on his knees. Sensor band 205D to be used as a hip sensor 202 has a sufficient length to encircle a patient's waist.

Once sensors 202 are placed in bands 205, each band may be placed on a body part, e.g., according to FIG. 10C. In some embodiments, shoulder band 205B may require connection of a hook and loop fastener. An elbow band 205 holding a sensor 202 should sit behind the patient's elbow. In some embodiments, hand sensor bands 205C may have one or more buckles to, e.g., fasten sensors 202 more securely, as depicted in FIG. 11B.

Each of sensors 202 may be placed at any of the suitable locations, e.g., as depicted in FIG. 10C. In some embodiments, sensors may be placed on ends of amputated limbs (e.g., “stumps”), prosthetic limbs, and/or end effectors. After sensors 202 have been placed on the body, they may be assigned or calibrated for each corresponding body part.

Generally, sensor assignment may be based on the position of each sensor 202. Sometimes, such as cases where patients have varying height discrepancies, assigning a sensor merely based on height is not practical. In some embodiments, sensor assignment may be based on relative position to, e.g., wireless transmitter module 202B.

FIG. 12 depicts an illustrative arrangement for various elements of a system, e.g., an HMD and sensors of FIGS. 10A-D. The arrangement includes one or more printed circuit boards (PCBs). In general terms, the elements of this arrangement track, model, and display a visual representation of the participant (e.g., a patient avatar) in the VR world by running software including the aforementioned VR application of HMD 201.

The arrangement shown in FIG. 12 includes one or more sensors 902, processors 960, graphic processing units (GPUs) 920, video encoder/video codec 940, sound cards 946, transmitter modules 910, network interfaces 980, and light emitting diodes (LEDs) 969. These components may be housed on a local computing system or may be remote components in wired or wireless connection with a local computing system (e.g., a remote server, a cloud, a mobile device, a connected device, etc.). Connections between components may be facilitated by one or more buses, such as bus 914, bus 934, bus 948, bus 984, and bus 964 (e.g., peripheral component interconnects (PCI) bus, PCI-Express bus, or universal serial bus (USB)). With such buses, the computing environment may be capable of integrating numerous components, numerous PCBs, and/or numerous remote computing systems.

One or more system management controllers, such as system management controller 912 or system management controller 932, may provide data transmission management functions between the buses and the components they integrate. For instance, system management controller 912 provides data transmission management functions between bus 914 and sensors 902. System management controller 932 provides data transmission management functions between bus 934 and GPU 920. Such management controllers may facilitate the arrangements orchestration of these components that may each utilize separate instructions within defined time frames to execute applications. Network interface 980 may include an ethernet connection or a component that forms a wireless connection, e.g., 802.11b, g, a, or n connection (WiFi), to a local area network (LAN) 987, wide area network (WAN) 983, intranet 985, or internet 981. Network controller 982 provides data transmission management functions between bus 984 and network interface 980.

Processor(s) 960 and GPU 920 may execute a number of instructions, such as machine-readable instructions. The instructions may include instructions for receiving, storing, processing, and transmitting tracking data from various sources, such as electromagnetic (EM) sensors 903, optical sensors 904, infrared (IR) sensors 907, inertial measurement units (IMUs) sensors 905, and/or myoelectric sensors 906. The tracking data may be communicated to processor(s) 960 by either a wired or wireless communication link, e.g., transmitter 910. Upon receiving tracking data, processor(s) 960 may execute an instruction to permanently or temporarily store the tracking data in memory 962 such as, e.g., random access memory (RAM), read only memory (ROM), cache, flash memory, hard disk, or other suitable storage component. Memory may be a separate component, such as memory 968, in communication with processor(s) 960 or may be integrated into processor(s) 960, such as memory 962, as depicted.

Processor(s) 960 may also execute instructions for constructing an instance of virtual space. The instance may be hosted on an external server and may persist and undergo changes even when a participant is not logged in to said instance. In some embodiments, the instance may be participant-specific, and the data required to construct it may be stored locally. In such an embodiment, new instance data may be distributed as updates that users download from an external source into local memory. In some exemplary embodiments, the instance of virtual space may include a virtual volume of space, a virtual topography (e.g., ground, mountains, lakes), virtual objects, and virtual characters (e.g., non-player characters “NPCs”). The instance may be constructed and/or rendered in 2D or 3D. The rendering may offer the viewer a first-person or third-person perspective. A first-person perspective may include displaying the virtual world from the eyes of the avatar and allowing the patient to view body movements from the avatar's perspective. A third-person perspective may include displaying the virtual world from, for example, behind the avatar to allow someone to view body movements from a different perspective. The instance may include properties of physics, such as gravity, magnetism, mass, force, velocity, and acceleration, which cause the virtual objects in the virtual space to behave in a manner at least visually similar to the behaviors of real objects in real space.

Processor(s) 960 may execute a program (e.g., the Unreal Engine or VR applications discussed above) for analyzing and modeling tracking data. For instance, processor(s) 960 may execute a program that analyzes the tracking data it receives according to algorithms described above, along with other related pertinent mathematical formulas. Such a program may incorporate a graphics processing unit (GPU) 920 that is capable of translating tracking data into 3D models. GPU 920 may utilize shader engine 928, vertex animation 924, and linear blend skinning algorithms. In some instances, processor(s) 960 or a CPU may at least partially assist the GPU in making such calculations. This allows GPU 920 to dedicate more resources to the task of converting 3D scene data to the projected render buffer. GPU 920 may refine the 3D model by using one or more algorithms, such as an algorithm learned on biomechanical movements, a cascading algorithm that converges on a solution by parsing and incrementally considering several sources of tracking data, an inverse kinematics (IK) engine 930, a proportionality algorithm, and other algorithms related to data processing and animation techniques. After GPU 920 constructs a suitable 3D model, processor(s) 960 executes a program to transmit data for the 3D model to another component of the computing environment (or to a peripheral component in communication with the computing environment) that is capable of displaying the model, such as display 950.

In some embodiments, GPU 920 transfers the 3D model to a video encoder or a video codec 940 via a bus, which then transfers information representative of the 3D model to a suitable display 950. The 3D model may be representative of a virtual entity that can be displayed in an instance of virtual space, e.g., an avatar. The virtual entity is capable of interacting with the virtual topography, virtual objects, and virtual characters within virtual space. The virtual entity is controlled by a user's movements, as interpreted by sensors 902 communicating with the VR engine. Display 950 may display a Patient View. The patient's real-world movements are reflected by the avatar in the virtual world. The virtual world may be viewed in the headset in 3D and monitored on the tablet in two dimensions. In some embodiments, the VR world is a game that provides feedback and rewards based on the patient's ability to complete activities. Data from the in-world avatar is transmitted from the HMD to the tablet to the cloud, where it is stored for later analysis. An illustrative architectural diagram of such elements in accordance with some embodiments is depicted in FIG. 13.

A VR system may also comprise display 970, which is connected to the computing environment via transmitter 972. Display 970 may be a component of a clinician tablet. For instance, a supervisor or operator, such as a therapist, may securely log in to a clinician tablet, coupled to the VR engine, to observe and direct the patient to participate in various activities and adjust the parameters of the activities to best suit the patient's ability level. Display 970 may depict at least one of a Spectator View, Live Avatar View, or Dual Perspective View.

In some embodiments, HMD 201 may be the same as or similar to HMD 1010 in FIG. 13. In some embodiments, HMD 1010 runs a version of Android that is provided by HTC (e.g., a headset manufacturer) and the VR application is an Unreal application, e.g., Unreal Application 1016, encoded in an Android package (.apk). The .apk comprises a set of custom plugins: WVR, WaveVR, SixenseCore, SixenseLib, and MVICore. The WVR and WaveVR plugins allow the Unreal application to communicate with the VR headset's functionality. The SixenseCore, SixenseLib, and MVICore plugins allow Unreal Application 1016 to communicate with the HMD accessory and sensors that communicate with the HMD via USB-C. The Unreal Application comprises code that records the position and orientation (P&O) data of the hardware sensors and translates that data into a patient avatar, which mimics the patient's motion within the VR world. An avatar can be used, for example, to infer and measure the patient's real-world range of motion. The Unreal application of the HMD includes an avatar solver as described, for example, below.

The operator device, clinician tablet 1020, runs a native application (e.g., Android application 1025) that allows an operator such as a therapist to control a patient's experience. Cloud server 1050 includes a combination of software that manages authentication, data storage and retrieval, and hosts the user interface, which runs on the tablet. This can be accessed by tablet 1020. Tablet 1020 has several modules.

As depicted in FIG. 13, the first part of tablet software is a mobile device management (MDM) 1024 layer, configured to control what software runs on the tablet, enable/disable the software remotely, and remotely upgrade the tablet applications.

The second part is an application, e.g., Android Application 1025, configured to allow an operator to control the software of HMD 1010. In some embodiments, the application may be a native application. A native application, in turn, may comprise two parts, e.g., (1) socket host 1026 configured to receive native socket communications from the HMD and translate that content into web sockets, e.g., web sockets 1027, that a web browser can easily interpret; and (2) a web browser 1028, which is what the operator sees on the tablet screen. The web browser may receive data from the HMD via the socket host 1026, which translates the HMD's native socket communication 1018 into web sockets 1027, and it may receive UI/UX information from a file server 1052 in cloud 1050. Tablet 1020 comprises web browser 1028, which may incorporate a real-time 3D engine, such as Babylon.js, using a JavaScript library for displaying 3D graphics in web browser 1028 via HTML5. For instance, a real-time 3D engine, such as Babylon.js, may render 3D graphics, e.g., in web browser 1028 on clinician tablet 1020, based on received skeletal data from an avatar solver in the Unreal Engine 1016 stored and executed on HMD 1010. In some embodiments, rather than Android Application 1026, there may be a web application or other software to communicate with file server 1052 in cloud 1050. In some instances, an application of Tablet 1020 may use, e.g., Web Real-Time Communication (WebRTC) to facilitate peer-to-peer communication without plugins, native apps, and/or web sockets.

The cloud software, e.g., cloud 1050, has several different, interconnected parts configured to communicate with the tablet software: authorization and API server 1062, GraphQL server 1064, and file server (static web host) 1052.

In some embodiments, authorization and API server 1062 may be used as a gatekeeper. For example, when an operator attempts to log in to the VR engine, the tablet communicates with the authorization server. This server ensures that interactions (e.g., queries, updates, etc.) are authorized based on session variables such as operator's role, the health care organization, and the current patient. This server, or group of servers, communicates with several parts of the VR engine: (a) a key value store 1054, which is a clustered session cache that stores and allows quick retrieval of session variables; (b) a GraphQL server 1064, as discussed below, which is used to access the back-end database in order to populate the key value store, and also for some calls to the application programming interface (API); (c) an identity server 1056 for handling the user login process; and (d) a secrets manager 1058 for injecting service passwords (relational database, identity database, identity server, key value store) into the environment in lieu of hard coding.

When the tablet requests data, it will communicate with the GraphQL server 1064, which will, in turn, communicate with several parts: (1) the authorization and API server 1062; (2) the secrets manager 1058, and (3) a relational database 1053 storing data for the VR engine. Data stored by the relational database 1053 may include, for instance, profile data, session data, game data, and motion data.

In some embodiments, profile data may include information used to identify the patient, such as a name or an alias. Session data may comprise information about the patient's previous sessions, as well as, for example, a “free text” field into which the therapist can input unrestricted text, and a log 1055 of the patient's previous activity. Logs 1055 are typically used for session data and may include, for example, total activity time, e.g., how long the patient was actively engaged with individual activities; activity summary, e.g., a list of which activities the patient performed, and how long they engaged with each on; and settings and results for each activity. Game data may incorporate information about the patient's progression through the game content of the VR world. Motion data may include specific range-of-motion (ROM) data that may be saved about the patient's movement over the course of each activity and session, so that therapists can compare session data to previous sessions' data. In some embodiments, file server 1052 may serve the tablet software's website as a static web host.

While the foregoing discussion describes exemplary embodiments of the present invention, one skilled in the art will recognize from such discussion, the accompanying drawings, and the claims, that various modifications can be made without departing from the spirit and scope of the invention. Therefore, the illustrations and examples herein should be construed in an illustrative, and not a restrictive sense. The scope and spirit of the invention should be measured solely by reference to the claims that follow.

1. A method of animating an avatar performing an activity in virtual reality, the method comprising:
accessing avatar skeletal data;
identifying a missing limb in the first skeletal data;
accessing a set of movement rules corresponding to the activity;
generating simulated full limb data based on the set of movement rules and the avatar skeletal data; and
rendering the avatar skeletal data with the simulated full limb skeletal data.

2. The method of claim 1, wherein the set of movement rules comprises symmetry rules.

3. The method of claim 2, wherein generating the simulated full limb data based on the set of movement rules and the avatar skeletal data further comprises generating the simulated full limb skeletal data based on reflecting position data for a full limb over an axis.

4. The method of claim 1, wherein the set of movement rules comprises predefined position rules.

5. The method of claim 4, wherein the generating simulated full limb data based on the set of movement rules and the avatar skeletal data further comprises generating the simulated full limb skeletal data based on a predefined position for the activity.

6. The method of claim 1, wherein the set of movement rules comprises prop position rules.

7. The method of claim 6, wherein the generating simulated full limb data based on the set of movement rules and the avatar skeletal data further comprises generating the simulated full limb skeletal data based on a relational position for a full limb.

8. The method of claim 1, wherein the avatar skeletal data is based on received position and orientation data for a plurality of body parts.

9. The method of claim 1, wherein the rendering the avatar skeletal data with the simulated full limb data comprises overriding a portion of the avatar skeletal data with the simulated full limb data.

10. The method of claim 1, wherein the accessing the set of movement rules corresponding to the activity comprises determining a movement pattern associated with the activity and accessing the set of movement rules corresponding to the movement pattern.

11.-26. (canceled)

27. A method of providing virtual reality therapy for an amputee, comprising:
receiving movement data of an intact limb and an amputated limb;
predicting synchronous movements based on the movement data of the intact limb; and
generating an avatar for the amputee based on the synchronous movements in place of the amputated limb.

28. The method of claim 27, wherein the predicting the synchronous movements is based on a relation between the intact limb and the amputated limb.

29. The method of claim 28, wherein the relation is a tether, a prop, or a symmetry between the two limbs that allows the position and orientation of one limb to determine the position and orientation of a partner limb.

30. The therapeutic activity of claim 27, wherein generating the avatar comprises a virtual image, a virtual reality image, or an augmented reality image.

31. A method for overriding an end effector for generating an avatar of a user, comprising:
collecting position and orientation data for a first limb of the user;
generating a virtual prop with a first contact region and a second contact region;
determining a position and orientation of the first contact region with the first limb; and
solving a position of a second limb based on the second contact region.

32. The method of claim 31, wherein the end effector of the second limb is overridden by the second contact region.

33. The method of claim 31, wherein the virtual prop extends in a direction that is perpendicular to the first limb.

34. The method of claim 31 further comprising assigning the position and orientation data of the first limb, or portion thereof, as an end effector, and solving a position and orientation of the first limb from the end effector.

35. The method of claim 31, wherein each contact region of the virtual prop is animated as hand grip or foot placement position.

36. The method of claim 31, wherein the first contact region and second contact region are connected by a tether that is at least one of the following: rigid, flexible, and stretchable.

37. The method of claim 36, wherein a constraint between both contact regions and the tether permits only an angle of between 0 and 45 degrees to form between the tether and at least one of the following: the first contact region and the second contact region.
